(19) United States
(12) Patent Application Publication
(10) Pub. No.: US 2006/0020029 A1
(43) Pub. Date: Jan. 26, 2006

(54) Pharmaceutical compositions from ethnobotanicals

(76) Inventors: Craig D. Shimasaki, Edmond, OK (US); Joshua O. Ojwang, Edmond, OK (US)

Correspondence Address: FULBRIGHT & JAWORSKI L.L.P., 600 CONGRESS AVE., SUITE 2400, AUSTIN, TX, 78701, US

(21) Appl. No.: 11/157,129

(22) Filed: Jun. 20, 2005

Related U.S. Application Data
(60) Provisional application No. 60/585,117, filed on Jul. 2, 2004.

(51) Int. Cl. C07C 69/88; A61K 31/235

(52) U.S. Cl. 514/533; 560/67; 560/69

(57) ABSTRACT

This invention relates to the field of drug discovery. Specifically, it describes a method (“Inverted Drug Screening” or “IDS™”) of identifying therapeutics from ethnobotanical (EB) preparations by repeatedly fractionating and testing fractions from EB sources. One aspect of the invention relates to quinic acid derivatives (e.g., derivatives of 3,5-dicaffeoyl quinic acid) for the treatment of respiratory syncytial virus (RSV) infection.

The present application claims benefit of priority to U.S. Provisional Application Ser. No. 60/585,117, filed Jul. 2, 2004, the entire contents of which are hereby incorporated by reference in their entirety.

The government owns rights in the present invention pursuant to grant number R43 AI46848 and R44 AI46848 from the National Institutes of Health SBIR program.

### BACKGROUND OF THE INVENTION

1. Field of the Invention

The present invention relates generally to the field of drug discovery. More particularly, the present invention relates to the identification of compounds from ethnobotanical preparations for the treatment of disease states.

2. Description of Related Art

Drug development typically involves the identification of a molecular target. Novel chemical entities are then synthesized to affect the function of the molecular target, and these chemical entities are ultimately tested clinically in human subjects. However, compounds that appear promising in pre-clinical trials often fail in clinical trials due to differences in the model system or animal used to test the compound, as compared to humans (e.g., differences in adsorption, excretion, metabolism, or toxicity). Other times the putative drug target proves to not be as relevant to the disease state as originally believed. Thus, this traditional approach to drug discovery exhibits significant limitations.

In contrast, several clinically successful drugs have been isolated from plants. For example, aspirin was discovered from the bark of the willow tree, and modifications to salicylate compounds have produced many new non-steroidal anti-inflammatory drugs. Codeine, morphine, and papaverine are alkaloids that were isolated from the juice of the opium poppy. Taxol was discovered in the bark of the Pacific Yew tree and is currently approved for use in treating ovarian and breast cancer. Vincristine and vinblastine were identified from extracts of the periwinkle plant and have been important in the treatment of leukemia and lymphoma. Digitoxin and other digitalis glycosides were discovered from the leaves of the foxglove plant and are used in the treatment of congestive heart failure. Although these clinically important drugs have been identified from plants, the tremendous variety of plants available for testing can present a daunting task for thorough investigation.

One way to narrow the field of investigation in the search for therapeutic compounds is to limit evaluation to natural products obtained from ethnobotanical (EB) sources historically reported to exhibit medicinal value. For example, quinine, which is used to treat malaria, was derived from certain cinchona barks which were previously used to treat malaria. Additionally, the National Cancer Institute has screened a large library of extracts taken from plants that have been used medicinally in an attempt to discover improved treatments for cancer and HIV based on the idea that ethnobotanicals might yield new therapeutics (Cragg et al., 1994). For example, application US2002/0111382 describes an application of isolating compounds from EB sources to treat viral infections. Nonetheless, a need exists for improved methods for the isolation and characterization of therapeutic substances from EB sources.

### SUMMARY OF THE INVENTION

There, in accordance with the present invention, there is provided a method of identifying a therapeutic composition comprising: identifying a substance that has been used as an ethnobotanical remedy for a given disease state, subjecting the substance to fractionation, testing one or more of the fractions in a screen for activity against the given disease state, and identifying one or more fractions that exhibit activity against the disease state, wherein an identified the fraction that exhibit activity comprises a therapeutic composition against the given disease state. The fractionation may comprise one or more of freezing, grinding, solubilizing, extracting, eluting, filtering, precipitating, partitioning or chromatography. The disease state may comprise an infectious disease, a genetic disease, cancer. The infectious disease may be a viral disease, a bacterial disease or a fungal disease. The disease state may also comprise a disease selected from the group consisting of cardiac disease, diabetes, a neurodegenerative disease, a viral disease, a fungal disease, a bacterial disease, and cancer. The ethnobotanical may comprise a quinic acid derivative. The testing may comprise an in vitro assay. In certain embodiments, the method may comprise identifying an active compound in the fraction.

Another aspect of the present invention relates to a purified compound having the structure:

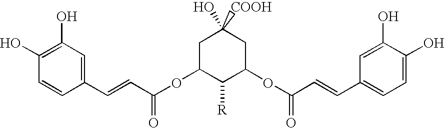

wherein R is selected from the group consisting of: alkyl, alkenyl, alkynyl, alkoxy, alkylaryl, halogen, hydrogen, trihalomethyl, NO₂, thioether, amine, SH, NH₂, —OCH₃, —OCH₂ (CH₂)ₙ CH₃, —(CH₂)ₙ OH, —(CH₂)ₙ NH₂, —N(CH₂)ₙ OH, —OCOOC(CH₃)₃, and an amino acid, and wherein n=0, 1, 2, 3, 4, or 5. R may be a halogen, preferably —F or —Cl. R may also be, in preferred embodiments, SH, NH₂, —OCH₃, —OCH₂ (CH₂)ₙ CH₃, —(CH₂)ₙ OH, —(CH₂)ₙ NH₂, —N(CH₂)ₙ OH, or —OCOOC(CH₃)₃; wherein n=0,1,2,3,4, or 5.

Another aspect of the present invention involves a method of inhibiting respiratory syncytial virus (RSV)-induced cytotoxicity comprising contacting a cell infected with RSV with the compound. The cell is may be located in an animal subject. The animal may be a human or non-human animal; in certain non-limiting embodiments, the animal is a cow, a pig, a horse, a rat, a dog, a cat, a rat or a mouse. The compound may be administered to the animal intranasally, intradermally, intraarterially, intraperitoneally, intralesionally, intracranially, intraarticularly, intraprostaticaly, intrapleurally, intratracheally, intranasally, intravitreally, intravaginally, intrarectally, topically, intratumorally, intramuscularly, intraperitoneally, subcutaneously, subconjunctival, intravesicularlly, mucosally, intrapericardially, intraumbilically, intraocularally, orally, topically, locally, inhalation (e.g., aerosol inhalation), injection, infusion, continuous infusion, localized perfusion bathing target cells directly, via a catheter, via a lavage, in cremes, in lipid compositions (e.g., liposomes), or by other method or any combination of the forgoing as would be known to one of ordinary skill in the art. The compound is preferably administered in a pharmaceutically acceptable carrier, diluent or vehicle. In certain preferred embodiments, a second anti-viral composition may be administered to the animal.

Another aspect of the present invention related to a method of inhibiting respiratory syncytial virus (RSV)-induced cytotoxicity comprising contacting a cell infected with RSV with a compound having the structure of a quinic acid derivative of the compound. The cell is preferably in an animal subject. The compound may be administered to the animal intranasally, intradermally, intraarterially, intraperitoneally, intralesionally, intracranially, intraarticularly, intraprostaticaly, intrapleurally, intratracheally, intranasally, intravitreally, intravaginally, intrarectally, topically, intratumorally, intramuscularly, intraperitoneally, subcutaneously, subconjunctival, intravesicularlly, mucosally, intrapericardially, intraumbilically, intraocularally, orally, topically, locally, inhalation (e.g., aerosol inhalation), injection, infusion, continuous infusion, localized perfusion bathing target cells directly, via a catheter, via a lavage, in cremes, in lipid compositions (e.g., liposomes), or by other method or any combination of the forgoing as would be known to one of ordinary skill in the art. The compound is preferably administered in a pharmaceutically acceptable carrier, diluent or vehicle. In certain preferred embodiments, a second anti-viral composition may be administered to the animal.

### DESCRIPTION OF ILLUSTRATIVE EMBODIMENTS

Although great steps have been taken in recent years to improve the ability to treat infectious disease, cancer and heart disease, the ability to intervene in these pathologic conditions is still limited by the battery of drugs available to the physician. Thus, drug discovery continues to be of paramount importance to the healthcare industry. Unfortunately, this is typically a laborious procedure, requiring massive screening projects to rule out millions of therapeutically irrelevant compounds. As such, there remains a need to streamline the drug discovery process.

The present invention addresses this problem by providing methods of identifying pharmaceuticals from EB preparations. In order to isolate and purify compounds from EB preparations, a method applies a systematic approach to isolating and identifying therapeutic compounds from EB preparations. First, an EB candidate composition is identified, where suitable screening procedures are available. Second, the composition is subjected to fractionation using any of a variety of means, including different combinations of physical, thermal, chemical and enzymatic fractionation. Third, the resulting fractions are tested for their utility against the selected disease state in appropriate screening methods, including in vitro, in cyto, and/or in vivo tests. Each EB fraction exhibiting activity is repeatedly subfractionated to reduce the number of candidate compounds. Ultimately, the therapeutically-relevant compounds present in the original EB preparation are substantially purified to the point where they can be identified.

Using this method, several compounds useful for treating infection by respiratory syncytial virus (RSV) have already been identified. Thus, in another aspect of the invention, these compounds and their use in inhibiting RSV infection are provided.

### A. ETHNOBOTANICAL PREPARATIONS

Throughout the past several thousand years, human cultures have utilized plants for a variety of purposes, including food, the production of clothing and shelter, and the treatment of medical ailments. Indeed, an Indian medical system called the Ayurveda was developed over 5000 years ago and codified the knowledge about the medicinal value of plants; even today, this system is used in India and elsewhere. A thousand years subsequently, Assyrians listed over 250 plants with medicinal uses, and ancient Sumerians are reported to have created a list of over 1000 medicinal plants, including drawings of opium poppy capsules (Principe, 1991). The Greek physician Hippocrates employed between 300 and 400 kinds of plants for the treatment of various ailments. Indeed, virtually all modern human cultures have a history of using plants for the treatment of disease states, including South American, Native American, Indian, and Chinese cultures.

This knowledge has often been passed on to subsequent generations via the written and/or spoken word. This knowledge was first termed “ethnobotany” in 1896 by Dr. John W. Harshberger, which he defined as “the study of plants used by primitive and aboriginal people”. Subsequently, in 1941, “ethnobotany” was described as “the study of the interrelationships of primitive men and plants”, and later, in 1967, the definition was expanded to “the relationships between man and his ambient vegitation” (Cotton, 1996). “Ethnobotanical” (“EB”) is defined here as referring to any preparation or fraction that has been produced using vegetative matter from at least one source which is historically reported to exhibit medicinal value. EB preparations can include vegetative matter from one or more plant sources. EB preparations can be obtained from places including herbal medicine pharmacies in the USA and China. This invention relates to the isolation and characterization of compounds from EB sources for the treatment of disease states. In particular, this invention provides a method of isolating pharmaceuticals from EB preparations.

B. INVERTED DRUG SCREENING (IDS™) SYSTEM SCREENING PROCESS

The present invention defines a method of “Inverted Drug Screening” (IDS™) for the identification of fractions of EB preparations, and compounds within EB fractions, for treating a disease state. IDS™ can be further applied to purify, isolate, and identify specific molecules for both the treatment of a disease state and the synthesis of other molecules for the treatment of a disease state. IDS™ first utilizes a repeated process of fractionation of EB preparations and in vitro and/or in vivo testing; as the EB fractions are fractionated and tested repeatedly, and samples are selected for further fractionation and testing based on their efficacy and potency in treating a disease state, the therapeutic compounds are further purified. The final step of IDSm involves the identification of specific compounds from the EB fractions, and chemical modifications of these compounds can be performed to increase their clinical utility to treat a disease.

I. Fractionation of EB Using IDS™

The first step of IDS™ involves the fractionation of EB preparations. Methods of fractionation include physical separation, chemical separation, chromatography, and thermal separation. Each of these methods is described in detail below.

a. Physical Separation

Fractionation using IDS™ can occur by methods of physical separation. These methods include, but are not limited to: grinding, sonicating, homogenizing, filtering, centrifuging, and precipitating. For example, an EB preparation may be ground into a powder for subsequent analysis; other examples include the centrifugation of a liquid EB preparation to separate the aqueuos and solid components of the preparation.

b. Chemical Separation

Fractionation using IDS™ can also occur by methods of chemical separation. These methods include, but are not limited to solubilizing (e.g., using hexane, chloroform, ethanol, water, or other solvent), extracting, eluting, and precipitating. For example, the use of various salts and solvents may be used to induce precipitation of substance(s) in a solution containing an EB preparation or fraction.

c. Chromatographic Separation Procedures

Chromatographic techniques may be employed to effect separation of EB preparations or fractions. Any of a wide variety of chromatographic procedures may be employed according to the present invention. For example, thin layer chromatography, column chromatography (e.g., using sephadex or cellulose in the column), exclusion chromatography, gas chromatography, high performance liquid chromatography, ion-exchange and molecular sieve chromatography, paper chromatography, adsorption chromatography, capillary electrophoresis, gel purification, polyacrylamide gel electrophoresis, isoelectric focusing, affinity chromatography, or supercritical flow chromatography may be used to effect separation of various chemical species. Chromatography may be employed with combinations of similar and/or other techniques.

Partition chromatography is based on the theory that if two phases are in contact with one another, and if one or both phases constitute a solute, the solute will distribute itself between the two phases. Usually, partition chromatography employs a column, which is filled with a sorbent and a solvent. The solution containing the solute is layered on top of the column. The solvent is then passed through the column, continuously, which permits movement of the solute through the column material. The solute can then be collected based on its movement rate. The two most common types of partition chromatograph are paper chromatograph and thin-layer chromatograph (TLC); together these are called adsorption chromatography. In both cases, the matrix contains a bound liquid. Other examples of partition chromatography are gas-liquid and gel chromatography.

Paper chromatography is a variant of partition chromatography that is performed on cellulose columns in the form of a paper sheet. Cellulose contains a large amount of bound water even when extensively dried. Partitioning occurs between the bound water and the developing solvent. Frequently, the solvent used is water. Usually, very small volumes of the solution mixture to be separated is placed at top of the paper and allowed to dry. Capillarity draws the solvent through the paper, dissolves the sample, and moves the components in the direction of flow. Paper chromatograms may be developed for either ascending or descending solvent flow. Two dimensional separations are permitted by changing the axis of migration 90° after the first run.

Thin layer chromatography (TLC) is very commonly used to separate lipids and, therefore, is considered a preferred embodiment of the present invention. TLC has the advantages of paper chromatography, but allows the use of any substance that can be finely divided and formed into a uniform layer. In TLC, the stationary phase is a layer of sorbent spread uniformly over the surface of a glass or plastic plate. The plates are usually made by forming a slurry of sorbent that is poured onto the surface of the gel after creating a well by placing tape at a selected height along the perimeter of the plate. After the sorbent dries, the tape is removed and the plate is treated just as paper in paper chromatography. The sample is applied and the plate is contacted with a solvent. Once the solvent has almost reached the end of the plate, the plate is removed and dried. Spots can then be identified by fluorescence, immunologic identification, counting of radioactivity, or by spraying varying reagents onto the surface to produce a color change.

In Gas-Liquid chromatography (GLC), the mobile phase is a gas and the stationary phase is a liquid adsorbed either to the inner surface of a tube or column or to a solid support. The liquid usually is applied as a solid dissolved in a volatile solvent such as ether. The sample, which may be any sample that can be volatized, is introduced as a liquid with an inert gas, such as helium, argon or nitrogen, and then heated. This gaseous mixture passes through the tubing. The vaporized compounds continually redistribute themselves between the gaseous mobile phase and the liquid stationary phase, according to their partition coefficients.

The advantage of GLC is in the separation of small molecules. Sensitivity and speed are quite good, with speeds that approach 1000 times that of standard liquid chromatography. By using a non-destructive detector, GLC can be used preparatively to purify grams quantities of material. The principal use of GLC has been in the separation of alcohols, esters, fatty acids and amines.

Gel chromatography, or molecular sieve chromatography, is a special type of partition chromatography that is based on molecular size. The theory behind gel chromatography is that the column, which is prepared with tiny particles of an inert substance that contain small pores, separates larger molecules from smaller molecules as they pass through or around the pores, depending on their size. As long as the material of which the particles are made does not adsorb the molecules, the sole factor determining rate of flow is the size. Hence, molecules are eluted from the column in decreasing size, so long as the shape is relatively constant. Gel chromatography is unsurpassed for separating molecules of different size because separation is independent of all other factors such as pH, ionic strength, temperature, etc. There also is virtually no adsorption, less zone spreading and the elution volume is related in a simple matter to molecular weight.

The gel material for gel chromatography is a three-dimensional network whose structure is usually random. The gels consist of cross-linked polymers that are generally inert, do not bind or react with the material being analyzed, and are uncharged. The space filled within the gel is filled with liquid and this liquid occupies most of the gel volume. Common gels are dextran, agarose and polyacrylamide; they are used for aqueous solution.

High Performance Liquid Chromatography (HPLC) is characterized by a very rapid separation with extraordinary resolution of peaks. This is achieved by the use of very fine particles and high pressure to maintain and adequate flow rate. Separation can be accomplished in a matter of minutes, or a most an hour. Moreover, only a very small volume of the sample is needed because the particles are so small and close-packed that the void volume is a very small fraction of the bed volume. Also, the concentration of the sample need not be very great because the bands are so narrow that there is very little dilution of the sample.

Affinity Chromatography is a chromatographic procedure that relies on the specific affinity between a substance to be isolated and a molecule that it can specifically bind to. This is a receptor-ligand type interaction. The column material is synthesized by covalently coupling one of the binding partners to an insoluble matrix. The column material is then able to specifically adsorb the substance from the solution. Elution occurs by changing the conditions to those in which binding will not occur (alter pH, ionic strength, temperature, etc.). The matrix should be a substance that itself does not adsorb molecules to any significant extent and that has a broad range of chemical, physical and thermal stability. The ligand should be coupled in such a way as to not affect its binding properties. The ligand should also provide relatively tight binding. And it should be possible to elute the substance without destroying the sample or the ligand. One of the most common forms of affinity chromatography is immunoaffinity chromatography.

d. Thermal Separation

Fractionation using IDS™ can also be performed using methods of thermal separation including, but not limited to, boiling, volatilizing, freezing, and flash-freezing. Thermal separation takes advantage of differences in the melting point and/or boiling point of compounds; for example, compounds that are in a specific states (i.e., gas or vapor, solid, liquid or fluid) can often be easily separated from other compounds that are in a different state.

II. In vitro, in cyto, and in vivo Testing Using IDS™

An important step of IDS™ involves testing of EB fractions. Several approaches can be utilized to test EB fractions and/or compounds from EB fractions, including the use of in vitro, in cyto, and/or in vivo assays.

a. In vitro assays

A quick, inexpensive and easy assay to run is an in vitro assay. Such assays generally use isolated molecules, can be run quickly and in large numbers, thereby increasing the amount of information obtainable in a short period of time. A variety of vessels may be used to run the assays, including test tubes, plates, dishes and other surfaces such as dipsticks or beads. An example of an in vitro assay is a binding assay.

b. In cyto Assays

A more typical approach to evaluating the properties of EB fractions and/or compounds from EB fractions is the use of in cyto testing. Thus, in one aspect, the present invention contemplates the screening of EB fractions and/or compounds from EB fractions for their ability to modulate mRNA expression and or protein accumulation of selected targets in a cell. Measuring the concentraion or biometabolism of selected metabolites in cells that may reflect various responses to a disease state in vivo. Other examples of in vitro tests that can be used to evaluate the properties of the EB fraction include, but are not limited to, the use of various cell lines (e.g., Hep-2 cells, Vero cells, MDCK cells) to evaluate cytotoxicity, viral plaque reduction, and the viral yield assay.

Depending on the assay, culture may be required. The cell is examined using any of a number of different physiologic assays. Alternatively, molecular analysis may be performed, for example, looking at protein expression, mRNA expression (including differential display of whole cell or polyA RNA) and others.

The exact parameters of an in cyto assay will vary depending on the disease state of interest. For example, in the search for novel anti-cancer therapeutics, an in cyto assay might involve the use of different cancer cell lines to determine the effects of a compound from an EB fraction on cell proliferation or expression of a specific gene, such as c-myc.

The present invention describes specific in cyto assays that can be used to evaluate novel compounds for treatment of viral diseases; these assays include cytotoxicity evaluation, viral plaque reduction, and the viral yield assay. Other in cyto assays that may also be used include the evaluation of the response of various cell lines to different viruses in the presence or absence of an EB fraction and/or compound from an EB fraction; using this approach, the ability of the virus to replicate or the expression of specific genes can be analyzed.

In other instances, novel anti-fungal agents may be desired. In these instances, in cyto assays that would be useful include the evaluation of the response of specific fungi grown in culture to the candidate EB fractions and/or compounds from EB sources. Using these in cyto assays, the rate of fungi growth or gene expression could be evaluated. In other instances, to evaluate compounds from EB sources on protozoan diseases, specific kinds of protozoan cells may be utilized to test how compounds from EB sources affect protozoan survival or gene expression.

If novel therapeutics for the treatment of a neurodegenerative disease are desired, then in cyto assays using cultured neurons can be employed. For example, testing the toxicity of MPTP on cultured neurons in the presence or absence of EB fractions or compounds from EB fractions might be a useful in cyto assay for obtaining novel therapeutics for the treatment of Parkinson's disease.

If novel therapeutics for the treatment of diabetes are instead desired, then other in cyto assays can be employed. For example, cultured cell lines from the islets of Langerhans could be tested in the presence or absence of EB fractions or compounds from EB fractions.

Novel therapeutics from EB sources may also be desired for the treatment of cardiac diseases. In these instances, in cyto tests using cultured cardiac cells or cultured explants of cardiac cells may be employed. Changes in gene expression may be monitored in response to EB fractions and/or compounds from EB sources. In some instances, it may be efficacious to utilize an entire heart taken from a non-human animal (e.g., a rat) to determine the acute effects of a specific compound on cardiac function.

Many different techniques can be applied to analysis of cell lines in in cyto assays; specifically, these techniques include northern blots, southern blots, PCR, real time (RT)-PCR, molecular beacon hybridization, and gene array technology to determine changes in gene expression in cell lines. Additionally, techniques including immunoblotting and binding assays may be useful to evaluate changes in protein levels in cells. Other techniques such as fluorescence-activated cell sorting (FACS) may also be used to separate and count cells depending certain cellular events (e.g., apoptosis, necrosis, etc.).

C. IN VIVO ASSAYS

In vivo assays can be particularly useful when evaluating the function of EB fractions and/or compounds from EB fractions. In vivo assays involve the use of various animal models, including transgenic animals that have been engineered to have specific defects, or carry markers that can be used to measure the ability of a candidate substance to reach and effect different cells within the organism. “Wild-type” animals that have not been engineered to harbor any specific mutation(s) are also useful for in vivo testing, and these “wild-type” animals may provide significant advantage over transgenic animals, due to the significant costs associated with producing a transgenic animal. Many varieties of animals are suitable for in vivo assays, including rats (e.g., cotton rats), rabbits, hamsters, guinea pigs, gerbils, woodchucks, cats, dogs, sheep, goats, pigs, cows, horses and monkeys (including chimps, gibbons and baboons). Assays for the properties (e.g., toxicity evaluation, ability to treat a disease state) may be conducted using an animal from, or animal model derived from, any of these species.

In such assays, one or more EB fraction or compound(s) from EB fraction(s) are administered to an animal, and the ability of the EB fraction or compound(s) from EB fraction(s) to affect a disease in the model, as compared to a similar animal not treated with the EB fraction or compound(s) from EB fraction(s), is evaluated. The characteristics may be any of those discussed above with regard to the function of a particular compound (e.g., enzyme, receptor, hormone) or cell (e.g., growth, tumorigenicity, survival), or response to a disease state (e.g., evaluation of viral titer), or instead a broader indication such as behavior, anemia, immune response, etc. Additionally, “add back” or “reconstitution” experiments may be performed to identify multiple compounds that work together (e.g., additively or synergistically) to produce a desired effect (e.g., amelioration of symptoms of a disease); in these experiments, combinations of identified compounds may be tested simultaneously to determine which combination of compounds are desirable or required to produce a desired effect.

Treatment of these animals with test compounds will involve the administration of the compound, in an appropriate form, to the animal. Administration will be by any route that could be utilized for clinical or non-clinical purposes, including but not limited to oral, nasal, buccal, or even topical. Alternatively, administration may be by intratracheal instillation, bronchial instillation, intradermal, subcutaneous, intramuscular, intraperitoneal or intravenous injection. Specifically contemplated routes are systemic intravenous injection, regional administration via blood or lymph supply, or directly to an affected site.

Determining the effectiveness of an EB fraction or compound(s) from EB fraction(s) in vivo may involve a variety of different criteria. Also, measuring toxicity and dose response can be performed in animals in a more meaningful fashion than in in vitro or in cyto assays.

III. Identification of compounds from EB fractions using IDS™

The final step of IDS™ involves identifying specific compounds from EB fractions. This aspect of the invention includes a method for the purification and identification of individual compounds from EB fractions using techniques including, but not limited to, HPLC (as described above), mass spectroscopy (MS), and nuclear magnetic resonance (NMR).

Where the term “substantially purified” is used, this designation will refer to a composition in which the compound forms the major component of the composition, such as constituting about 50%, about 60%, about 70%, about 80%, about 90%, about 95% or more of the compound in the EB fraction.

a. Mass Spectroscopy

Mass spectrometry provides a means of “weighing” individual molecules by ionizing the molecules in vacuo and making them “fly” by volatilization. Under the influence of combinations of electric and magnetic fields, the ions follow trajectories depending on their individual mass (m) and charge (z). For low molecular weight molecules, mass spectrometry has been part of the routine physical-organic repertoire for analysis and characterization of organic molecules by the determination of the mass of the parent molecular ion. In addition, by arranging collisions of this parent molecular ion with other particles (e.g., argon atoms), the molecular ion is fragmented forming secondary ions by the so-called collision induced dissociation (CID). The fragmentation pattern/pathway very often allows the derivation of detailed structural information. Other applications of mass spectrometric methods in the known in the art can be found summarized in Methods in Enzymology, 1990.

Two ionization/desorption techniques are electrospray/ionspray (ES) and matrix-assisted laser desorption/ionization (MALDI). ES mass spectrometry was introduced by Fenn et al., 1984; WO 90/14148 and its applications are summarized in review articles (Smith et al., 1990; Ardrey, 1992). As a mass analyzer, a quadrupole is most frequently used. The determination of molecular weights in femtomole amounts of sample is very accurate due to the presence of multiple ion peaks which all could be used for the mass calculation.

b. Nuclear Magnetic Resonance (NMR)

NMR provides a method for determining the molecular structure of a compound. NMR can is typically performed using liquid-state samples of a composition, although solid-state samples can also be used. NMR takes advantage of the fact that certain atoms (e.g., hydrogen) are intrinsically magnetic. The spinning of the positively charged proton generates a magnetic moment. When the atom is exposed to an external magnetic field, the spinning will orient itself with regard to the magnetic field in one of two orientations (called α and β states). The α state is slightly lower in energy compared to the β state, and the energy difference between these states is proportional to the strength of the imposed magnetic field. A transition from the α state to the β state occurs when a nucleus adsorbs electromagnetic radiation of the appropriate frequency. Because the flow of electrons around a magnetic nucleus generates a small local magnetic field, this local magnetic field can oppose (or “shield”) nearby nuclei. Consequently, nuclei in different environments adsorb energy at different frequencies of electromagnetic radiation. The adsorption of electromagnetic radiation across a variety of frequencies can be measured, and this information can be used to determine the structure of the compound that comprises the sample.

C. Disease States

Disease states include infectious diseases (e.g. a viral, bacterial, protozoan, or fungal disease), genetic diseases, diabetes, neurodegenerative diseases, and cancer. Disease states may affect any human or non-human animal.

I. Fungal Diseases

There is reason to believe that compounds from ethnobotanical sources will be useful for the treatment of fungal infections; for example, extracts from certain Hawaiian medicinal plants have inhibited the growth of fungi in vitro (Locher et al., 1995). Fungal diseases are caused by fungal and other mycotic pathogens (some of which are described in Human Mycoses (Beneke, 1979); Opportunistic Mycoses of Man and Other Animals (Smith, 1989); and Scripp's Antifungal Report, 1992); fungal diseases range from mycoses involving skin, hair, or mucous membranes, such as, but not limited to, Aspergillosis, Black piedra, Candidiasis, Chromomycosis, Cryptococcosis, Onychomycosis, or Otitis externa (otomycosis), Phaeohyphomycosis, Phycomycosis, Pityriasis versicolor, ringworm, Tinea barbae, Tinea capitis, Tinea corporis, Tinea cruris, Tinea favosa, Tinea imbricata, Tinea manuum, Tinea nigra (palmaris), Tinea pedis, Tinea unguium, Torulopsosis, Trichomycosis axillaris, White piedra, and their synonyms, to severe systemic or opportunistic infections, such as, but not limited to, Actinomycosis, Aspergillosis, Candidiasis, Chromomycosis, Coccidioidomycosis, Cryptococcosis, Entomophthoramycosis, Geotrichosis, Histoplasmosis, Mucormycosis, Mycetoma, Nocardiosis, North American Blastomycosis, Paracoccidioidomycosis, Phaeohyphomycosis, Phycomycosis, pneumocystic pneumonia, Pythiosis, Sporotrichosis, and Torulopsosis, and their synonyms, some of which may be fatal.

Known fungal and mycotic pathogens include, but are not limited to, Absidia spp., Actinomadura madurae, Actinomyces spp., Allescheria boydii, Alternaria spp., Anthopsis deltoidea, Apophysomyces elegans, Arnium leoporinum, Aspergillus spp., Aureobasidium pullulans, Basidiobolus ranarum, Bipolaris spp., Blastomyces dermatitidis, Candida spp., Cephalosporium spp., Chaetoconidium spp., Chaetomium spp., Cladosporium spp., Coccidioides immitis, Conidiobolus spp., Corynebacterium tenuis, Cryptococcus spp., Cunninghamella bertholletiae, Curvularia spp., Dactylaria spp., Epidermophyton spp., Epidermophyton floccosum, Exserophilum spp., Exophiala spp., Fonsecaea spp., Fusarium spp., Geotrichum spp., Helminthosporium spp., Histoplasma spp., Lecythophora spp., Madurella spp., Malassezia furfur, Microsporum spp., Mucor spp., Mycocentrospora acerina, Nocardia spp., Paracoccidioides brasiliensis, Penicillium spp., Phaeosclera dematioides, Phaeoannellomyces spp., Phialemonium obovatum, Phialophora spp., Phoma spp., Piedraia hortai, Pneumocystis carinii, Pythium insidiosum, Rhinocladiella aquaspersa, Rhizomucor pusillus, Rhizopus spp., Saksenaea vasiformis, Sarcinomyces phaeomuriformis, Sporothrix schenckii, Syncephalastrum racemosum, Taeniolella boppii, Torulopsosis spp., Trichophyton spp., Trichosporon spp., Ulocladium chartarum, Wangiella dermatitidis, Xylohypha spp., Zygomyetes spp. and their synonyms. Other fungi that have pathogenic potential include, but are not limited to, Thermomucor indicae-seudaticae, Radiomyces spp., and other species of known pathogenic genera. These fungal organisms are ubiquitous in air, soil, food, decaying food, etc. Histoplasmoses, Blastomyces, and Coccidioides, for example, cause lower respiratory infections. Trichophyton rubrum causes difficult to eradicate nail infections. In some of the patients suffering with these diseases, the infection can become systemic causing fungal septicemia, or brain/meningal infection, leading to seizures and even death.

II. Viral Diseases

There is also reason to believe that compounds found in ethnobotanical sources will be useful for treating viral infection; for example, U.S. Pat. Nos. 5,494,661 and 5,211,944 describe proanthocyanidin polymers which were derived from an EB source and may be useful in the treatment of a respiratory virus. Viral diseases include, but are not limited to: influenza A, B and C, parainfluenza (including types 1, 2, 3, and 4), paramyxoviruses, Newcastle disease virus, measles, mumps, adenoviruses, adenoassociated viruses, parvoviruses, Epstein-Barr virus, rhinoviruses, coxsackieviruses, echoviruses, reoviruses, rhabdoviruses, lymphocytic choriomeningitis, coronavirus, polioviruses, herpes simplex, human immunodeficiency viruses, cytomegaloviruses, papillomaviruses, virus B, varicella-zoster, poxviruses, rubella, rabies, picomaviruses, rotavirus, Kaposi associated herpes virus, herpes viruses type 1 and 2, hepatitis (including types A, B, and C), and respiratory syncytial virus (including types A and B).

III. Bacterial Diseases

There is reason to believe that compounds from ethnobotanical sources will be useful for the treatment of bacterial infections; for example, extracts from certain Hawaiian medicinal plants have inhibited the growth of bacteria in vitro (Locher et al., 1995). Bacterial diseases include, but are not limited to, infection by the 83 or more distinct serotypes of pneumococci, streptococci such as S. pyogenes, S. agalactiae, S. equi, S. canis, S. bovis, S. equinus, S. anginosus, S. sanguis, S. salivarius, S. mitis, S. mutans, other viridans streptococci, peptostreptococci, other related species of streptococci, enterococci such as Enterococcus faecalis, Enterococcus faecium, Staphylococci, such as Staphylococcus epidermidis, Staphylococcus aureus, particularly in the nasopharynx, Hemophilus influenzae, pseudomonas species such as Pseudomonas aeruginosa, Pseudomonas pseudomallei, Pseudomonas mallei, brucellas such as Brucella melitensis, Brucella suis, Brucella abortus, Bordetella pertussis, Neisseria meningitidis, Neisseria gonorrhoeae, Moraxella catarrhalis, Corynebacterium diphtheriae, Corynebacterium ulcerans, Corynebacterium pseudotuberculosis, Corynebacterium pseudodiphtheriticum, Corynebacterium urealyticum, Corynebacterium hemolyticum, Corynebacterium equi, etc. Listeria monocytogenes, Nocordia asteroides, Bacteroides species, Actinomycetes species, Treponema pallidum, Leptospirosa species and related organisms. The invention may also be useful against gram negative bacteria such as Klebsiella pneumoniae, Escherichia coli, Proteus, Serratia species, Acinetobacter, Yersinia pestis, Francisella tularensis, Enterobacter species, Bacteriodes and Legionella species and the like.

IV. Protozoan Diseases

Additionally, there is reason to believe that compounds from ethnobotanical sources will also be useful for the treatment of protozoan or macroscopic diseases; for example, the drug emetine was derived from an ethnobotanical source and has been used to treat Amoebic dysentery (Wolfe, 1982). Protozoan or macroscopic diseases include infection by organisms such as Cryptosporidium, Isospora belli, Toxoplasma gondii, Trichomonas vaginalis, Cyclospora species, for example, and for Chlamydia trachomatis and other Chlamydia infections such as Chlamydia psittaci, or Chlamydia pneumoniae, for example.

D. DRUG FORMULATIONS AND METHOD OF TREATING RSV INFECTIONS

Using the IDS™ approach to drug screening aspect of this invention, several compounds have been isolated and identified that will be used to treat disease states. An aspect of the invention involves the isolation and identification of purified dicaffeoyl-quinic acids useful in treating RSV infections.

In particular, infection by the respiratory syncytial virus (RSV) is one example of a disease state that could benefit from the development of new drugs from EB sources. RSV is the leading cause of severe lower respiratory tract infections in infants and children under 2 years of age (Parrot et al., 1973; Glezen et al., 1982; Chanock and McInosh, 1990). Overall, this virus has been reported to be responsible for 40-50% of hospitalizations for bronchiolitis in the United States and 25% of pediatric hospitalizations for pneumonia. Recently, RSV has also been identified as a cause of lower respiratory tract infections in adults (Dowell et al., 1996). Currently, only two drugs are approved for treatment of RSV infection: RespiGam™ and Synagis®, both of which are antibody preparations. Synagis® is the preferred treatment and may cost at least $5000 for a regimen. Due to the continued medical impact of RSV infection and limitations associated with current therapies, a need exists for improved treatments for RSV. Thus, EB sources may yield new, more effective therapies for treatment of RSV infection, and this invention provides a method for isolating and characterizing novel compounds from EB sources.

One aspect of the present invention involves the isolation of certain quinic acid derivatives from EB preparations for the treatment of RSV infection. Another aspect of the present invention involves certain quinic acid derivatives for the treatment of RSV infection.

I. Chemical Definitions

As used herein, the term “acyl” or “acyl group” is to be understood to mean groups including alkyl and aryl subunits.

An “alkyl” group refers to a saturated aliphatic hydrocarbon, including straight-chain, branched chain, and cyclic alkyl groups. Alkyl groups can comprise any combination of acyclic and cyclic subunits. Further, the term “alkyl” as used herein expressly includes saturated groups as well as unsaturated groups. Unsaturated groups contain one or more (e.g., one, two, or three), double-bonds and/or triple-bonds. The term “alkyl” includes substituted and unsubstituted alkyl groups. When substituted, the substituted group(s) may be hydroxyl, cyano, alkoxy, ═O, ═S, NO₂, N(CH₃)₂, amino, or SH. Specifically, the alkyl group has 1 to 12 carbons. More specifically, it is a lower alkyl of from 1 to 7 carbons.

An “alkenyl” group refers to an unsaturated hydrocarbon group containing at least one carbon-carbon double-bond, including straight-chain, branched-chain, and cyclic groups. Specifically, the alkenyl group has 1 to 12 carbons. More specifically, it is a lower alkenyl of from 1 to 7 carbons, more preferably 1 to 4 carbons. The alkenyl group may be substituted or unsubstituted. When substituted, the substituted group(s) is preferably hydroxyl, cyano, alkoxy, ═O, ═S, NO₂, N(CH₃)₂, halogen, amino, or SH.

An “alkynyl” group refers to an unsaturated hydrocarbon group containing at least one carbon-carbon triple bond, including straight-chain, branched chain, and cyclic groups. Specifically, the alkynyl group has 1 to 12 carbons. More specifically, it is a lower alkynyl of from 1 to 7 carbons, more Specifically 1 to 4 carbons. The alkynyl group may be substituted or unsubstituted. When substituted, the substituted group(s) is preferably hydroxyl, cyano, alkoxy, ═O, ═S, NO₂, N(CH₃)₂, amino, or SH.

An “alkoxy” group refers to an “—O-alkyl” group, where “alkyl” is defined above.

An “aryl” group refers to an aromatic group which has at least one ring having a conjugated pi electron system, and includes carbocyclic aryl, heterocyclic aryl, and biaryl groups, all of which may be optionally substituted. Specifically, the aryl is a substituted or unsubstituted phenyl or pyridyl. Specifically aryl substituent(s) are halogen, trihalomethyl, hydroxyl, SH, OH, NO₂, amine, thioether, cyano, alkoxy, alkyl, and amino groups.

An “alkylaryl” group refers to an alkyl (as described above), covalently joined to an aryl group (as described above). Specifically, the alkyl is a lower alkyl. “Carbocyclic aryl” groups are groups wherein the ring atoms on the aromatic ring are all carbon atoms. The carbon atoms are optionally substituted with specific groups as described for aryl groups above.

“Heterocyclic aryl” groups are groups having from 1 to 3 heteroatoms as ring atoms in the aromatic ring and the remainder of the ring atoms are carbon atoms. Siutable heteroatoms include oxygen, sulfur, and nitrogen, and include furanyl, thienyl, pyridyl, pyrrolyl, N-lower alkyl pyrrolo, pyrimidyl, pyrazinyl, imidazoyl, and the like, all optionally substituted.

An “amide” refers to a —C(O)—NH—R, where R is either alkyl, aryl, alkylaryl, or hydrogen.

A “thioamide” refers to a —C(S)—NH—R, where R is either alkyl, aryl, alkylaryl, or hydrogen.

An “ester” refers to a —C(O)—OR′, where R′ is either alkyl, aryl, or alkylaryl.

An “amine” refers to a —N(R″)R′″, where R″ and R′″ is each independently either hydrogen, alkyl, aryl, or alkylaryl, provided that R″ and R′″ are not both hydrogen.

A “thioether” refers to —S—R, where R is either alkyl, aryl, or alkylaryl.

A “sulfonyl” refers to —S(O)₂—R, where R is aryl, C(CN)═C-aryl, CH2-CN, alkylaryl, NH-alkyl, NH-alkylaryl, or NH-aryl.

II. Quinic Acid Derivatives: Anti-RSV Agents

The present invention presents the isolation of certain quinic acid derivatives that display efficacy in treating RSV infection, including 3,5-di-O-caffeoylquinic acid (3,5-DCQA), from EB preparations. Additionally, in certain specific embodiments, subsequent quinic acid derivatives can be obtained and/or synthesized and tested for the ability to inhibit RSV infection.

Quinic acid derivatives can be grouped as mono-(type I), di-(type II) and tri-O-substitute (type III) quinic acids. The following diagram illustrates a generic structure for quinic acid derivatives:

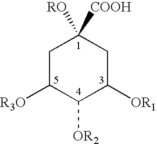

wherein R and R₁—R₃ are each independently chosen from the group consisting of hydrogen and acyl.

Using the above generic structure for quinic acid derivatives, type I, type II, and type III quinic acid derivatives can be defined as follows. Below each category of quinic acid derivatives are sub-groups within that type; for example, “Ia” refers to type Ia quinic acid derivatives.

Type I:

| | |
|---|---|
| Ia | R = R₁ = R₂ = H, R₃ = acyl |
| Ib | R = R₁ = R₃ = H, R₂ = acyl |
| Ic | R = R₂ = R₃ = H, R₁ = acyl |
| Id | R = acyl, R₁ = R₂ = R₂ = H |

Type II:

| | |
|---|---|
| IIa | R = R₁ = H, R₂ = R₃ = acyl |
| IIb | R = R₂ = H, R₁ = R₃ = acyl |
| IIc | R = R₃ = H, R₁ = R₂ = acyl |
| IId | R₁ = R₂ = H, R = R₃ = acyl |
| IIe | R₁ = R₃ = H, R = R₂ = acyl |
| IIf | R₁ = R₂ = H, R = R₃ = acyl |
| IIg | R = R₁ = acyl, R₂ = R₃ = H |

Type III:

| | |
|---|---|
| IIIa | R = R₁ = R₂ = acyl, R₃ = H |
| IIIb | R = R₁ = R₃ = acyl, R₂ = H |
| IIIc | R = R₂ = R₃ = acyl, R₁ = H |
| IIId | R = H, R₁ = R₂ = R₃ = acyl |

Examples of compounds of Type IIb quinic acid derivatives include the following structures:

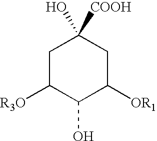

wherein R₁ and R₃ are each independently acyl.
1. R₁=R₃=caff
2. R₁=R₃=ac-caff
3. R₁=R₃=Me-caff
4. R₁=R₃=cou
5. R₁=R₃=ac-cou
6. R₁=R₃=Me-cou
7. R₁=R₃=cinn
8. R₁=R₃=succinyl
9. R₁=R₃=isobutyryl
10. R₁=caff, R₃=cou
11. R₁=caff, R₃=ac-cou
12. R₁=caff, R₃=Me-cou
13. R₁=caff, R₃=cinn
14. R₁=caff, R₃=succinyl
15. R₁=caff, R₃=isobutyryl

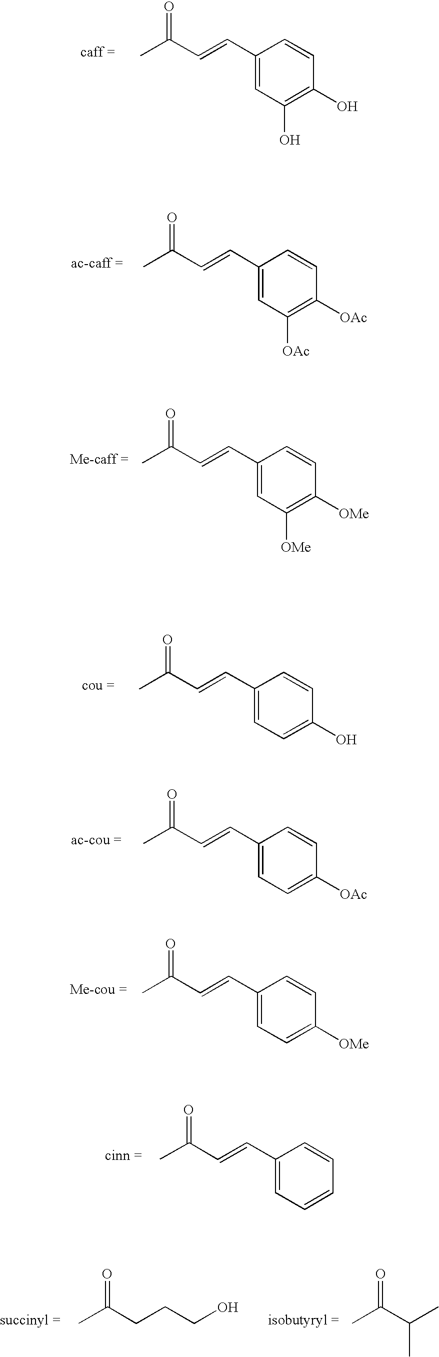

One particular Type IIb quinic acid derivative, 3,5-dicaffeoyl quinic acid (3,5-DCQA, listed as compound #1 above for the Type IIb quinic acid derivatives), has been identified by the inventors using IDS™ for the treatment of respiratory syncytial virus infections. Additionally, the inventors identified a potential problem with using 3,5-DCQA; specifically, when 3,5-dicaffeoyl quinic acid is contained in a protic solvent, this compound will undergo a transesterification reaction resulting in 4,5-dicaffeoyl quinic acid. 4,5-dicaffeoyl quinic acid has been identified by the inventors using IDS™ as being useful for treating respiratory syncytial virus; however, this compound has also been shown by the inventors to be less potent than 3,5-DCQA for the treatment of respiratory syncytial virus.

The inventors have discovered that transesterification of 3,5-DCQA in a protic solvent presents a significant problem for the administration of 3,5-DCQA. To overcome this problem, either an aprotic solvent (or other medium) may be used for administration of 3,5-DCQA or derivatives of 3,5-DCQA may be also be used. Because the use of certain aprotic solvents (e.g., DMSO) for the administration of 3,5-DCQA acid may result in toxicity to the subject, the use of derivatives of 3,5-DCQA is preferred.

The inventors have identified several 3,5-DCQA derivatives that resist transesterification in protic solvents. The following derivatives of 3,5-dicaffeoyl quinic acid may be used:

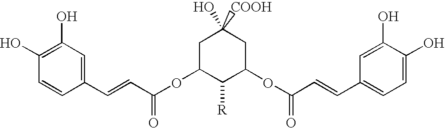

wherein R is chosen from the group consisting of alkyl, alkenyl, alkynyl, alkoxy, alkylaryl, halogen, hydrogen, trihalomethyl, NO₂, thioether, amine, SH, NH₂, —OCH₃, —OCH₂ (CH₂)ₙ CH₃, —(CH₂)ₙ OH, —(CH₂)ₙ NH₂, —N(CH₂)ₙ OH, —OCOOC(CH₃)₃, and an amino acid, wherein n=0,1,2,3,4, or 5. In certain preferred embodiments, R is F, Cl, SH, NH2, —OCH₃, —OCH₂ (CH₂)ₙ CH₃, —(CH₂)ₙ OH, —(CH₂)ₙ NH_{2,} —N(CH₂)ₙ OH, —OCOOC(CH₃)₃, or an amino acid, wherein n=0,1,2,3,4, or 5.

Another aspect of the invention involves a method of inhibiting RSV-induced cytotoxicity comprising contacting a cell infected with RSV with a quinic acid derivative. The cell may exist in a human or non-human animal, and compound may be administered intranasally, intradermally, intraarterially, intraperitoneally, intralesionally, intracranially, intraarticularly, intraprostaticaly, intrapleurally, intratracheally, intranas ally, intravitreally, intravaginally, intrarectally, topically, intratumorally, intramuscularly, intraperitoneally, subcutaneously, subconjunctival, intravesicularlly, mucosally, intrapericardially, intraumbilically, intraocularally, orally, topically, locally, inhalation (e.g., aerosol inhalation), injection, infusion, continuous infusion, localized perfusion bathing target cells directly, via a catheter, via a lavage, in cremes, in lipid compositions (e.g., liposomes), or by other method or any combination of the forgoing as would be known to one of ordinary skill in the art (see, for example, Remington's Pharmaceutical Sciences, 18th Ed. Mack Printing Company, 1990, incorporated herein by reference). The compound may also be administered in a pharmaceutically acceptable carrier, diluent or vehicle. The method includes administering to the animal a second anti-viral composition.

The phrases “pharmaceutical or pharmacologically acceptable” refer to molecular entities and compositions that do not produce an adverse, allergic or other untoward reaction when administered to an animal, such as, for example, a human, as appropriate. The preparation of an pharmaceutical composition that contains at least one dicaffeoyl quinic acid (e.g., a 3,5-dicaffeoyl quinic acid derivative) or additional active ingredient will be known to those of skill in the art in light of the present disclosure, as exemplified by Remington's Pharmaceutical Sciences, 18th Ed. Mack Printing Company, 1990, incorporated herein by reference. Moreover, for animal (e.g., human) administration, it will be understood that preparations should meet sterility, pyrogenicity, general safety and purity standards as required by FDA Office of Biological Standards. As used herein, “pharmaceutically acceptable carrier” includes any and all solvents, dispersion media, coatings, surfactants, antioxidants, preservatives (e.g., antibacterial agents, antifungal agents), isotonic agents, absorption delaying agents, salts, preservatives, drugs, drug stabilizers, gels, binders, excipients, disintegration agents, lubricants, sweetening agents, flavoring agents, dyes, such like materials and combinations thereof, as would be known to one of ordinary skill in the art (see, for example, Remington's Pharmaceutical Sciences, 18th Ed. Mack Printing Company, 1990, pp. 1289-1329, incorporated herein by reference). Except insofar as any conventional carrier is incompatible with the active ingredient, its use in the therapeutic or pharmaceutical compositions is contemplated.

The actual dosage amount of a composition of the present invention administered to an animal patient can be determined by physical and physiological factors such as body weight, severity of condition, the type of disease being treated, previous or concurrent therapeutic interventions, idiopathy of the patient and on the route of administration. The practitioner responsible for administration will, in any event, determine the concentration of active ingredient(s) in a composition and appropriate dose(s) for the individual subject.

### E. EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Fractionation of EB Preparations

The four individual preparations (Table 1), which are constituents of EB preparation ZX-2187P, were obtained and evaluated separately to determine the active constituent. This eliminated three quarters of the excess materials to be analyzed. Next, the active EB preparations selected after the initial anti-RSV screening of the crude EB preparation extracts (using the in vitro CPE assay system described below) were subjected to chemical extraction and fractionation to obtain partially purified fractions used in these studies. The extraction was performed as follows:

The herbal product was finely ground using a Cuisinart Coffee Mill as described above. The finely ground material (10 g) was packed into a cellulose extraction thimble (33 mm×118 mm, Whatman) and placed in a Soxhtlet Apparatus connected to an Allihn Condenser. A 500-ml round bottom flask was used for the solvent extraction. The powder was continuously extracted with 150-200 ml of a solvent for 10-24 hours. The solvents, chosen in order of use, were hexane (to extract any non-functionalized hydrophobic compounds), chloroform (to continue the extraction of other non-functionalized hydrophobic compounds), absolute ethanol (to remove all compounds soluble in a amphiphilic solvent), and water (to extract all aqueous soluble compounds). Water extraction was performed by steeping the previously extracted powder (with hexane, chloroform and ethanol) in boiling water for 15 min and filtering using a wetted filter paper. All extracts were evaporated under vacuum.

To differentiate the partially purified fractions from the parent EB preparation, the first 2 or 3 letters of extracting solvent were added in parenthesis after EB preparation designation. For example, the alcohol soluble fraction of ZX-2227P was designated ZX-2227P (alc), etc.

The fractions obtained here were completely soluble in the appropriate solvents, which allowed us to use relatively exact weights in the biological assays. The solubility of these fractions in water was relatively good, to the extent that most stock solutions of the fractions were made up in water prior to testing. Each fraction was evaluated for anti-RSV activity using the CPE assay as described below. The fraction(s) which showed a significant anti-RSV effect were studied further.

### Example 2

### Evaluation of EB Fractions Using In Vitro Antiviral and Cytotoxicity Testing

The materials and methods used for in vitro antiviral evaluation of EB fractions are as follows. Hep-2 cells and RSV (Long strain) were obtained from ATCC. The cell line was propagated in MEM supplemented with 10% heat-inactivated fetal bovine serum (FBS) containing 100 μg/ml of antibiotics (streptomycin/penicillin). The stock preparation of RSV (Long strain) was obtained from the supernatant of infected culture showing 100% cytopathic effect (CPE) and clarified by low speed centrifugation to remove cell debris. The viral stock was stored at −135° C. The antiviral effect of EB fractions were investigated using a CPE assay method as follows: Cells (4×10⁴/well) were seeded in 96-well plates and incubated overnight at 37° C. in a humidified 5% Co₂ atmosphere. The overnight medium (MEM containing 10% FBS) was removed, and wells were rinsed twice with 1× phosphate buffer saline (PBS, 200 μl) before adding 10×TCID₅₀ of RSV suspension in fresh medium (MEM plus 2% FBS) to each well, except for cell only wells. After 2 h of virus adsorption at 37° C. in a humidified 5% Co₂ atmosphere, the medium was removed and the cells were rinsed once with 100 μl of 1×PBS. Varying concentrations of each EB fraction (or ribavirin as a positive control) in culture medium (MEM plus 2% FBS, 200 μl ) were added to the appropriate wells, and plates were incubated for 7 days at 37° C. in a humidified 5% CO₂ atmosphere. Each concentration of the EB fractions or ribavirin was tested in triplicate wells. CellTiter 96 Aqueous Non-Radioactive Cell Proliferation Assay (Promega) was used to monitor the antiviral activity following the manufacturer's instruction. After incubation, the cells were rinsed twice with 200 μl of 1×PBS prior to the addition of 100 μl of fresh medium (MEM plus 10% FBS). Twenty microliters of MTS reagent (Promega) were added to each well, and the plates were incubated at 37° C. in a humidified 5% CO₂ atmosphere until significant color development was observed (1 to 4 h). The extent of cell viability was measured at 490 nm using an ELISA reader, and the absorbance was used to calculate percent inhibition. Antiviral activity of each EB fraction or ribavirin is expressed as IC₅₀, which represents the concentration of the test substance required to reduce the virus infection (as measured by the destruction of the host cells) by 50%.

The materials and methods used for in vitro cytotoxicity evaluation of EB fractions are as follows. The toxicity of the EB fractions to host cells was also tested in parallel with the antiviral testing and was conducted as follows: Uninfected Hep-2 or Vero cells were resuspended in medium (MEM supplemented with 10% FBS). The viable cells (determined by trypan blue staining) were dispensed into 96-well (1×10⁴ cells/well) microtiter plates. Varying concentrations of each EB fraction or ribavirin (in medium, 20 μl) were added to the appropriate wells, and the plates were incubated at 37° C. in a humidified 5% CO₂ atmosphere for 7 days. Each concentration of the EB fractions or ribavirin was assayed in triplicate wells. The cytotoxicity of each concentration of EB fractions or ribavirin was determined using the CellTiter 96 Aqueous Non-Radioactive Cell Proliferation (MTS) Assay as described above. The average absorbance for each concentration of EB fractions or ribavirin was tabulated, and the data obtained were used to calculate CC₅₀ (concentration of test article required to inhibit the growth of host cells by 50%).

Investigation of the Antiviral and Cytotoxicity Properties of EB Fractions was performed as follows using RSV-Long. This part of the study served as the primary screen for anti-RSV activity of EB preparations before any further studies were considered. Five of the most effective and least cytotoxic EB preparations in the initial screen of over fifty EB preparations are listed in Table 1 below. The EB preparation ZX-2187P, which was the main herbal medicine of the Phase I grant, is a mixture of Coptidis Rhizoma (ZX-2199P), Sutellariae Radix (ZX-2200P), Phellodendri Cortex (ZX-2201P) and Gardeniae Fructus (ZX-2202P) medicinal herbs. Based on favorable results using ZX-2187P, we decided to obtain its constituents and test them separately to identify the herb(s) responsible for the observed anti-RSV activity. In addition, in Phase I we selected. and tested five more EB preparations (ZX-2226P to ZX-2231P, listed in Table 1) based on the anti-RSV activity and cytotoxicity, as well as historical use to treat respiratory viral infections.

TABLE 1
List of Selected ER preparations against RSV-long

| | | | CC₅₀ | |
|---|---|---|---|---|
| | | | for | TI |
| | | IC₅₀ | Hep-2) | (IC₅₀/ |
| | | (μg/ml) | (μg/ml) | CC₅₀) |
| 1. ZX-2187P* | Huang Lian Jie Du Tang | 4.34 | 60 | 14 |
| a. ZX-2199P | Huang Lian (Coptidis | 3.4 | 15 | 4.4 |
| | Rhizoma) | | | |
| b. ZX-2200P | Huang Qin (Scutellariae | >100 | 119 | <1.2 |
| | Radix) | | | |
| c. ZX-2201P | Huang Bai (Phellodendri | 84 | 204 | <2.4 |
| | Cortex) | | | |
| d. ZX-2202P | Shen Zhi Zi (Fructus | 77 | >400 | >5.2 |
| | Garbeniae) | | | |
| 2. ZX-2226P | Dried Forsythia | 17 | >200 | >12 |
| 3. ZX-2227P | Flos Lonicerae | 35 | >300 | >8.6 |
| 4. ZX-2229P | Herba Taraxac | 23 | >100 | >4.3 |
| 5. ZX-2230P | Yu Xing Cao (Houttuynia | 52 | >200 | >3.8 |
| | Cordata) | | | |

*ZX.2187P is a blend of ZX-2199P, ZX-2200P, ZX-2201P, and ZX-2202P.

The results in Table 1 show that ZX-2187P (parent) and ZX-2199P have similar anti-RSV activity, but ZX-2199P was slightly more cytotoxic to Hep-2 cells than ZX-2187P. The preferential enrichment of cytotoxicity with the active constituent, using Hep-2 cells, indicates that one or more other constituents buffered the cytotoxicity effect of ZX-2199P in the whole preparation. Two other constituents, ZX-2201P and ZX-2202 were moderately active, and ZX-2200P was inactive up to 100 μg/ml. The EB preparations ZX-2226P, ZX-2227P, ZX-2229P and ZX-2230P had reasonable anti-RSV activity with no cytotoxicity observed with the highest concentration tested.

In conclusion, the anti-RSV activity observed with ZX-2187P appears to be almost exclusively due to contribution from ZX-2199P; therefore, ZX-2199P was selected instead of the parent ZX-2187P for further evaluation in spite of the reduction in therapeutic index. In addition, ZX-2226P, ZX-2227P, ZX-2229P and ZX-2230P were selected based on their anti-RSV activities and low cytotoxicity for further evaluation with the idea of isolating, identifying, and enhancing the active compounds via fractionation.

Investigation of the antiviral and cytotoxicity properties of EB fractions using two RSV strains was performed as follows. The EB preparations listed in Table 1 were partially purified as described above. The anti-RSV testing of the resultant fractions was conducted against RSV-Long and RSV B in two or more separate experiments as described above. In parallel, the cytotoxicity evaluation of these fractions was done in Hep-2 and Vero cells. In each experiment, each fraction was evaluated in a series of eight concentrations in triplicate. The end-points of antiviral and cytotoxicity effects were monitored by the CPE assay method. The averages of the IC₅₀ and CC₅₀ values from two or more separate experiments of the most active fractions are summarized in Table 2 below.

TABLE 2
Summary of IC₅₀, CC₅₀ and TI values for the selected EB extracted fractions*

| | RSV-Long in Hep-2 | RSV-B in Hep-2 cells | Hep-2 cells | Vero cells |
|---|---|---|---|---|
| | IC₅₀ (μg/ml) (TI) | IC₅₀ (μg/ml) (TI) | CC₅₀ (μg/ml) | CC₅₀ (μg/ml) |
| ZX-2187P (alc) | 3.2 ± 0.74 (3.5) | 1.8 ± 0.8 (6.2) | 11.1 ± 8.0 | 180.3 |
| ZX-2199P (aq) | 3.4 ± 0.65 (4.8) | 1.7 ± 0.5 (9.65) | 16.4 ± 11.5 | >250 |
| ZX-2226P (aq) | 20.1 ± 1.23 (20.45) | 19.1 ± 5.1 (21.5) | 411 | >250 |
| ZX-2226P (50%) | 16.8 ± 0.07 (10.14) | 6.7 ± 5 (25.4) | 170.4 ± 14.7 | >1000 |
| ZX-2227P (alc) | 4.7 ± 1.7 (53.19) | 2.8 ± 1.4 (89.3) | 250 | >1000 |
| ZX-2227P (50%) | 5.7 ± 4.3 (87.7) | 1.5 ± 0.5 (333) | 500 | >1000 |
| ZX-2229P (aq) | 19.0 ± 5.8 (35.15) | 23.6 ± 5.2 (28.3) | 667.9 | >250 |
| ZX-2230P (aq) | 10.2 ± 1.8 (>100) | 8.4 ± 3.3 (>100) | >1000 | >1000 |
| Ribavirin | 3.8 ± 0.2 (20.1) | 3.2 ± 0.5 (23.9) | 76.4 ± 4.4 | >100 |

*Average of two or three independent experiments. Each concentration was tested in triplicate wells.
The value in parentheses is the Therapeutic Index (TI) using Hep-2 cells, (alc) = absolute alcohol fraction, (50%) = fraction extracted with 50% alcohol in water and (aq) = aqueous fraction.

The data compiled in Table 2 show that most of the fractions listed have good inhibitory activities against both RSV-Long and B strains and exhibit low cytotoxicity in Hep-2 as well as Vero cells. Fractions ZX-2227P (alc), ZX-2227P (50%), ZX-2229P (aq) and ZX-2230P (aq) have TI values as good as or better than that for ribavirin. The fractions ZX-2187P (alc) and ZX-2199P (aq) exhibit high toxicity in Hep-2 cells compared to Vero cells. We enriched the anti-RSV activity of ZX-2227P ten-fold and ZX-2230P three-fold, respectively, upon fractionation. In contrast, the fractionation of ZX-2226P and ZX-2229 did not improve the activity. It was not surprising that the potency of ZX-2199P was not enhanced upon fractionation, because the active compounds were in aqueous solution, which was the solvent used in crude extraction.

In conclusion, the activity profiles of several of these extracted fractions are noteworthy when compared to the currently used anti-RSV therapeutic agent Ribavirin. Based on these data, ZX-2199P (aq), ZX-2227P (alc) and ZX-2230P (aq) were selected for further evaluation. The ZX-2199P (aq) fraction was selected instead of the fraction ZX-2187P (alc) from the parent EB preparation ZX-2187P for two reasons. First, the other three botanical constituents making up ZX-2187P were relatively inactive against RSV. This observation suggested that the anti-RSV activity observed with ZX-2187P came primarily from ZX-2199P. Secondly, a substantially larger amount of material could be obtained from the aqueous fraction of the ZX-2199P EB preparation than from ZX-2187P (alc).

### Example 3

### Evaluation of Specificity of Antiviral Properties of EB Fractions Using Influenza A Type 1 and Herpes Simplex Virus Type 1 and 2 Viruses

To determine whether the fractions are specific RSV inhibitors rather than general antiviral agents, the inventors tested them against influenza type A/Shangdong/09/93 (H3N2), herpes simplex type 1 (MacItyre), and herpes simplex type 2 (MS) using the CPE assay system. All three fractions were inactive against these viruses up to 500 μg/ml. In conclusion, these results suggest that the anti-RSV activity of these fractions is specific and warrant further investigation including testing in vivo.

### Example 4

### Antiviral Evaluation of EB Fractions Using the Plaque Reduction Assay (PRA) System

The materials and methods used for PRA are as follows. The PRA was employed to evaluate the effect of ZX-2199P (aq), ZX-2227P (alc), and ZX-2230P (aq) in another independent assay system. Briefly, Hep-2 or Vero cells were seeded at a cell density of 4×10⁴ cells/well in 12-well plates (MEM plus 10% FBS, 1 ml) and incubated overnight at 37° C. in a humidified 5% CO₂ atmosphere. The overnight medium was removed, and the wells were rinsed twice with 2 ml 1×PBS before adding 40-100 plaque forming units (pfu) of RSV suspension in fresh medium (MEM plus 2% FBS) to each well except cell only wells. After 2 h of virus adsorption, with interval tilting of the culture every 15 minutes to distribute inoculum evenly, the medium was removed and the cells were rinsed twice with 1 ml of 1×PBS. Two milliliters of varying concentrations of ZX-2199P (aq), ZX-2227P (alc), ZX-2230P (aq), or ribavirin in an agar plus culture medium (MEM plus 2% FBS) were added to the appropriate wells, and the plates were incubated as above. The overlay was prepared by mixing 1 part of 1.2% SeaPlaque and 1 part 2×MEM containing 4% FBS, plus appropriate concentrations of each EB fraction or ribavirin. Each concentration of the EB fraction or ribavirin was tested in duplicate wells. After 7 days, the agarose was carefully removed and cells were rinsed twice with 2 ml of PBS. The cell monolayer was fixed using 0.2% crystal violet in 20% methanol at room temperature. After 30 minutes, the fixer was removed by washing with deionized water, and the plates were air-dried prior to counting plaques. The number of plaques obtained for each fraction or ribavirin was expressed as a percentage of the control. The percentage values obtained were used to calculate IC₅₀ values.

TABLE 3
Average IC₅₀ values for the selected extracts against RSV-long in PRA*

| | RSV-Long in Hep-2 | Hep-2 cells | |
|---|---|---|---|
| Identification | IC50 (μg/ml) | CC50 (μg/ml) | TI |
| ZX-2199P (AQ) | 3.57 | 16.4 | 4.8 |
| ZX-2227P (alc) | 4.38 | 250 | 53 |
| ZX-2230P (AQ) | 10.91 | >1000 | >98 |
| RIBAVIRIN | 3.84 | 76.4 | 20 |

*Average of two or three or more independent experiments. Each concentration was tested in triplicate wells; (alc) = absolute alcohol fraction and (aq) = aqueous fraction.

The results in Table 3 show that selected EB fractions, ZX-2199P (aq), ZX-2227P (alc), and ZX-2230P (aq), inhibited RSV infection in this assay system to the same extent as observed using the CPE assay system. In conclusion, RSV inhibition by the EB fractions was confirmed using another independent assay system.

### Example 5

### Antiviral Evaluation of EB Fractions Using the Viral Yield Assay (VYA) System

The materials and methods used for VYA are as follows. Hep-2 cells were plated in 96-well plates at a cell density of 10,000 cells/well and incubated overnight at 37° C. in a humidified 5% CO₂ atmosphere. The overnight medium (MEM containing 10% FBS) was removed, and the wells were rinsed twice (PBS, 200 μl) before adding 1 to 100×TCID₅₀ of RSV suspension in fresh medium (MEM plus 2% FBS) to each well except for cell only wells. After 2 h of virus adsorption at 37° C. in a humidified 5% Co₂ atmosphere, the medium (MEM plus 2% FBS) was removed, and the cells were rinsed once with 100 μl of 1×PBS. Varying concentrations of each EB fraction or control drug in culture medium (MEM plus 2% FBS, 200 μl) were added to the appropriate wells, and the plates were incubated at 37° C. in a humidified 5% CO₂ atmosphere. Each concentration of the test articles or control drug was tested in triplicate wells. After 7 days, the cells were subjected to one cycle of freezing at −135° C. and thawing at 37° C. to disrupt cells. The aliquots from each of the eight wells of a given column were transferred to the first column of a fresh 96-well monolayer culture of Hep-2 cells. The contents were mixed and serially diluted across the remaining columns of the secondary plate. Each column of the original primary plate was diluted across a separate plate in this manner. The cultures were incubated for 7 additional days, and the end point was monitored by CPE assay as described above.

TABLE 4
Viral Yield Summary Table

| | ZX-2199P (aq) | | ZX-2227P (alc) | | ZX-22230P (aq) | | Ribaivrin | |
|---|---|---|---|---|---|---|---|---|
| Conc. | Mean Titer | Reduction | Mean Titer | Reduction | Mean Titer | Reduction | Mean Titer | Reduction |
| (μg/ml) | (log 10) | (log10) | (log 10) | (log10) | (log 10) | (log10) | (log 10) | (log10) |
| 0 | 4 | | 4 | | 4 | | 4 | |
| 3.1 | 2 | 2 | 1 | 3 | 2 | 2 | 3 | 1 |
| 25.0 | 0 | 4 | 0 | 4 | 0 | 4 | 2 | 2 |

Each concentration was tested in triplicate wells,
(alc) = absolute alcohol fraction and
(aq) = aqueous fraction

The VYA study data compiled in Table 4 show that ZX-2199P (aq) and ZX-2230P (aq) reduced the viral titer by 2- and 4-logs when tested at concentrations of 3.1 and 25 μg/ml, respectively. ZX-2227P (alc) at equivalent concentrations reduced the viral titers by 3- and 4-logs, respectively, while ribavirin, at equivalent concentrations, reduced the viral titer by only 1- and 2-logs, respectively. Although some of the effect of ZX-2199P (aq) at 25 μg/ml could be due to cytotoxicity against the host cells (CC₅₀=16 μg/ml) during the initial phase (seven days of viral replication and exposure to the test article), this should not be a significant factor for any of the other test articles.

In conclusion, the RSV inhibition by the EB fractions was confirmed using a third independent assay system. These EB fractions reduced the viral production at least 2-logs better than ribavirin. The results obtained from the in vitro studies provide strong evidence that the EB fractions are very potent inhibitors of RSV replication.

### Example 6

### Evaluate Antiviral Properties of EB Fractions In Vivo

Investigation of the antiviral activity and toxicity of selected EB preparations in cotton rats was performed as follows. The primary goal of these studies was to determine whether any of the anti-RSV activities demonstrated in vitro translated into activity in vivo.

The in vivo efficacy of the three lead EB fractions were evaluated in the cotton rat (CR) RSV infection model. Results of treatment with ZX-2199 (aq), ZX-2227P (alc), and ZX-2230P (aq) were compared to placebo and a positive control (ribavarin). All groups consisted of four randomly selected cotton rats. All test materials were administered intraperitoneally with once daily dosing. Dose and experimental schedule are shown in Table 5. The placebo group was administered distilled water at volumes and schedules identical to the other groups receiving investigational test articles. In general, timing of administration of ribavarin to the ribavarin-treated group paralleled that of the other treatment groups except that initiation of dosing was on Day 1 for Experiments 1 and 2 and Day 0 for Experiment 3. The dose of ribavarin was 90 mg/kg/day in Experiments 1 and 3 and 180 mg/kg/day in Experiment 2. All animals were inoculated intranasally with approximately 100 median cotton rat infectious doses (CRID₅₀) of RSV A2 on Day 0. Experiments were terminated on either Day 3 or 4 post-infection. On these days maximum RSV pulmonary titers are usually seen in untreated cotton rats. On the day that the experiment was terminated, all CRs were sacrificed using CO₂ to asphyxiate the animals. The lungs from the animals were subsequently removed. The lungs of sacrificed animals were removed, rinsed in sterile phosphate-buffered saline (PBS; pH 7.2) and weighed. Each set of lungs was then transpleurally lavaged using 3 ml of 2% FCS-MEM media. The resulting lung fractions were kept on ice until they were assayed for RSV. The lung fractions of animals from each group were tested for the levels of RSV present.

TABLE 5
In vivo experiment protocol summary

| | Dose Test | | | | | |
|---|---|---|---|---|---|---|
| | Article | | | | | |
| Experiment | (mg/kg/day) | Day -1 | Day 0 | Day 1, 2 | Day 3 | Day 4 |
| 1 | 100 | | Treat w/test article | Treat w/test | Treat w/test article | Harvest CRs |
| | | | Infect CRs 1 hr | article | | |
| | | | later | | | |
| 2 | 100 | Treat w/test | Treat w/test article | Treat w/test | Harvest CRs | |
| | | article | Infect CRs ½ hr | article | | |
| | | | later | | | |
| 3 | 250 | Treat w/test | Infect CRs | Treat w/test | Harvest CRs | |
| | | article | Treat w/test article | article | | |
| | | | ½ hr later | | | |

TABLE 6
Summary of in vivo results

| | Respiratory Syncytial Virus Pulmonary Titer in | | | | | | |
|---|---|---|---|---|---|---|---|
| | Cotton Rats (CR) values expressed as log 10 | | | | | | |
| | Experiment 1 | | Experiment 2 | | Experiment 3 | | |
| | Titers from | Mean | Titers from | Mean | Titers from | Mean | Overall |
| Treatment | individual CR | Titers ± SD | individual CR | Titers ± SD | individual CR | Titers ± SD | Mean Titer |
| Placebo | 5.3, 5.8, 5.8, 5.3 | 5.6 ± 0.29 | 4.8, 5.3, 4.3, 4.8 | 4.8 ± 0.4 | 4.3, 4.8, 4.8, 4.8, | 4.7 ± 0.2 | 5.03 ± 0.3 |
| ZX-2199P (aq) | 0.0, 4.8, 0.0, 3.8 | 2.2 ± 2.52 | 4.8, 4.3, 2.8, 2.8 | 3.7 ± 1.0 | 4.3, 0.0, 0.0, 0.0 | 1.1 ± 2.2 | 2.33 ± 1.9 |
| ZX-2227P (alc) | 5.3, 4.3, 4.8, 4.8 | 4.8 ± 0.4 | 3.8, 2.8, 2.8, 5.3 | 3.7 ± 1.2 | 0.0, 0.0, 3.8, 4.3 | 2.0 ± 2.3 | 3.50 ± 1.3 |
| ZX-2230P(aq) | 5.3, 3.8, 3.3, 3.8 | 4.1 ± 0.87 | 0.0, 0.0, 3.8, 3.3 | 1.8 ± 2.1 | 4.8, 3.8, 4.3, 4.8 | 4.4 ± 0.5 | 3.43 ± 1.2 |
| Ribavirin | 3.3, 2.8, 3.3, 2.8 | 3.1 ± 0.29 | 1.8, 2.3, 2.3, 2.8 | 2.3 ± 0.4 | 2.8, 0.0, 3.3, 2.8 | 2.2 ± 1.5 | 2.53 ± 0.73 |

Placebo = distilled water,
CR = cotton rat,
Titers are expressed in log 10.

The data from three experiments are presented in Table 6. In Experiment 1, virus was not detected in two animals treated with ZX-2199P (aq), and the other two virus titers were reduced by at least one log. In this experiment ZX-2199P (aq), ZX-2227P (alc), ZX-2230P (aq) and ribavirin reduced the mean viral titer by 3.4, 0.8, 1.5 and 2.5 logs, respectively.

In Experiment 2, no virus was detected in two animals treated with ZX-2230P (aq). The compounds tested, ZX-2199P (aq), ZX-2227P (alc), ZX-2230P (aq), and ribavirin, reduced the mean viral titer by 1.1, 1.1, 3.0 and 2.5 logs, respectively.

In Experiment 3, no virus was detected in three animals treated with ZX-2199P (aq) and two animals treated with ZX-2227P (alc). In this experiment the compounds tested, ZX-2199P (aq), ZX-2227P (alc), ZX-2230P (aq), and ribavirin, reduced the mean viral titer by 3.6, 2.7, 0.3 and 2.5 logs, respectively.

In all three experiments, the ability of the EB fractions to reduce viral titer can be observed. The overall mean viral titer showed that all the three fractions reduced the viral titers in the lungs of RSV infected animals by at least 1.5 logs. Of all the three EB fractions, ZX-2199P (aq) demonstrated the best activity. It reduced the overall mean viral titer in three experiments by 2.7 logs, which was better than ribavirin (2.5 logs). If the three experiments are combined, 5 out of 12, 2 out 12, and 2 out of 12 animals treated with ZX-2199P (aq), ZX-2227P (alc) and ZX-2230P (aq) had no detectable virus, respectively, whereas only 1 out of 12 ribavarin-treated animals appeared to be virus-free.

Toxicity studies were performed. In these studies, the weights of test animals were determined at the beginning and conclusion of each experiment. Each animal was also observed daily for morbidity, morality, diarrhea, or other untoward responses. No evidence of toxicity was observed on these animals treated with up to 250 mg/kg/day for 4 days.

In conclusion, these results support the use of EB fractions for treatment of RSV infection in vivo.

### Example 7

### Determine the Stage of Effect for EB Fractions

Determine the Time of Addition for EB Fractions. Compounds have a certain span of time when they are most active. This activity is related to the life cycle of the virus and the step in the cycle where the compounds act. In this example, experiments were performed to investigate the effect of the time of addition of EB fractionated products on anti-RSV activity. The activity profile was monitored using the CPE assay when EB fractions or ribavirin were added to Hep-2 cells at the start of infection (0), seven (+7), and twenty-six (+26) hours post-infection using 10×TCID₅₀ of RSV-Long.

TABLE 7
Time of Addition

| EB-Extract | | | |
|---|---|---|---|
| Fractions | IC₅₀ ± SD (μg/ml)* | | |
| I.D. | 0 h | +7 h | +26 h |
| ZX-2199P (aq) | 3.95 ± 1.7 | 3.23 ± 0.83 | 5.7 ± 4.8 |
| ZX-2227P (alc) | 5.3 ± 2.4 | 7.13 ± 3.3 | 8.45 ± 3.2 |
| ZX-2230P (aq) | 10.8 ± 1.8 | >50 | >50 |
| Ribavirin | 3.6 ± 0.36 | 3.9 ± 0.64 | 4.43 ± 0.88 |

*Serial dilution of the fraction or ribavirin in 96-well plates starting from either 50 or 100 μg/ml was performed. The average was calculated from 3 separate experiments; each concentration was tested in triplicate wells. Infection was determined by using the CPE assay.

Data from this study are summarized in Table 7. Like ribavirin, ZX-2199P (aq) and ZX-2227P (alc) could be added for at least 26 hours post-infection without compromising potency, while it was necessary to administer ZX-2230 (aq) within a few hours of virus addition.

Because a single complete life cycle for RSV is achieved within 18 to 24 h post-infection, these results suggest that ZX-2199P (aq) and ZX-2227P (alc) may be acting late in viral replication whereas ZX-2230P (aq) may be acting at a much earlier stage (e.g., initial viral attachment and/or cellular entry). Although these findings support the hypothesis that EB ZX-2230P (aq) inhibits RSV infection by a mechanism of action quite distinct from ZX-2199P (aq) and ZX-2227P (alc), they must be interpreted with some caution since CPE can only be observed after seven days which permits multiple rounds of viral replication in this assay system. Nonetheless, these results support the use of ZX-2230P (aq) in prophylaxis and ZX-2199P (aq) and ZX-2227P (alc) as both therapeutic and prophylactic agents.

Investigation of Viral Inactivation using EB Fractions. Some compounds have the ability to irreversibly bind to or totally inactivate the infectious nature of the RSV virion. To examine this possibility, evaluations of the reversibility and inactivation potential of the EB fractions were performed.

Selected EB fractions were added at an effective concentration to 100× virus concentrate and incubated at 4° C. At this pre-dilution step, each EB fraction concentration was 50 μg/ml and virus titer was 100×TCID₅₀. After 2 h, the infectivity of the virus was measured by diluting the virus 100-fold into fresh Hep-2 cells. This action resulted in a 100-fold dilution of the therapeutic products from 50 μg/ml to 0.5 μg/ml and the virus from 100× to 1×TCID₅₀. At this, the EB concentrations were below their effective anti-RSV concentration, unless they had irreversibly inactivated the virus prior to dilution. In contrast, the virus titer was still high enough to infect the cells. The MTS-based CPE assay system was used to measure infectivity.

No evidence of inactivation was detected. In conclusion, the EB fractions tested here inhibited RSV production by interfering with virus life cycle rather than directly inactivating the virus.

A limited pretreatment of host cells with EB fractions was investigated as follows. The MTS-based CPE assay was used to evaluate the activity of EB fractionated products or ribavirin when present only before infection. In this experiment, the therapeutics were added to an overnight confluent Hep-2 culture at various time points (i.e., −24, −4, −2 and 0 h) prior to viral infection. Just before viral addition (10×TCID₅₀), the medium was removed and the cells were rinsed twice with PBS to remove any residual test article. The cells were incubated in fresh medium without the test article. After 7 days, the viral inhibition was evaluated using CPE assay protocol.

TABLE 8
Effect of Limited Pretreatment on Anti-RSV Activity
of EB Fractionated Products

| | IC₅₀ (μg/ml) of Test Articles | | | |
|---|---|---|---|---|
| Pre-Treatment | ZX2199P | | ZX-2230P | |
| Time (hrs) | (aq) | ZX-2227P (alc.) | (aq) | Ribavirin |
| 24 | 32.50 | >100 | >100 | >50 |
| 8 | 40.63 | >100 | >100 | >50 |
| 4 | 49.25 | >100 | >100 | >50 |
| 0 | >100 | >100 | >100 | >50 |

Serial dilution of the fraction or ribavirin in 96-well plates starting from 100 μg/ml was performed. Each concentration was tested in triplicate wells. Infection was determined by using a CPE Assay.

The results compiled in Table 8 show that the ZX-2199P (aq) extract was the only test article that showed antiviral effect solely upon pre-treatment in two separate experiments. The most and least activity occurred when the ZX-2199P (aq) extract was added 24 h and 0 h pre-infection, respectively.

In conclusion, these data support a stage of effect wherein ZX-2199P (aq) either accumulates in the intracellular space during pretreatment and even upon removal is retained inside the cells at sufficient levels to prevent virus infection, or, alternatively, pretreatment alters the cells in such a way as to prevent RSV infection and/or replication. The other EB therapeutics and ribavirin had minimal effect, suggesting that the drugs must be present during the infection to prevent either viral entry or transmission between infected and non-infected cells.

Investigation of the Multiplicity of Infection (MOI) Variation Using EB Fractions. Certain compounds, which inhibit virus in cell cultures infected at low MOI, are often less active at higher virus-to-cell ratios. The effect of varying the multiplicity of infection of RSV was examined using RSV Long strain in a CPE assay system. In this study, Hep-2 cells were infected with RSV (Long strain) at various MOIs (1000×TCID₅₀, 100×TCID₅₀, and 10×TCID₅₀,). The level of viral inhibition by test drugs was monitored on day 7 post-infection.

The results in Table 9 show that the anti-RSV effect of EB therapeutics ZX-2199P (aq) and ZX-2227P (alc) is relatively insensitive to changes in viral MOI. Conversely, ZX-2230P (aq) has a 5-10 fold decrease in anti-RSV activity at 1000×TCID₅₀, consistent with the suggestion made above (see “Determine the Time of Addition for EB Fractions×) that the mechanism of action of ZX-2230P (aq) is distinct from the other EB fractions tested here.

TABLE 9
Effect of MOI variation

| | IC₅₀ (μg/ml) of Test Articles | | |
|---|---|---|---|
| EB Fraction I.D. | 1000 × TCID₅₀ | 100 × TCID₅₀ | 10 × TCID₅₀ |
| ZX-2199P (aq) | 3.13 | 4.76 | 6.25 |
| ZX-2227P (alc.) | 10.97 | 6.25 | 7.48 |
| ZX-2230P (aq) | 50 | 11.41 | 6.61 |

Serial dilution of the fraction in 96-well plates starting from 50 μg/ml was performed. Each concentration was tested in triplicate wells. Infection was determined by using a CPE Assay.

In conclusion, therapeutic benefit to be obtained from treatment with ZX-2230P may require that administration be initiated either prior to RSV infection or early post-infection before development of high respiratory tract titers. On the other hand, this limitation does not seem to apply to the active agent(s) in ZX-2199P or ZX-2227P so it may be of benefit to administer them even relatively late in the course of RSV infection.

### Example 8

### Determination of In Vitro Efficacy of an EB Fraction Using Analysis of Viral RNA

In this example, evaluation of the amount of viral RNA was used as an alternative approach for determination of the antiviral effects of EB fractions. The EB fraction ZX-2227P (alc) was selected because of its good anti-RSV activity and very low cytotoxicity.

The method for analysis of viral RNA is as follows. Six well plates were infected with 1 ml stock of RSV-Long (10×TCID₅₀) diluted 1:20 in infection medium (DMEM+2% FBS). The plates were incubated in 37° C. incubators for 60 min, after which the inoculum was removed and the plates were rinsed. Two milliliters of infection medium was added to appropriate wells, containing 1 or 20 μg/ml of ribavirin or ZX-2227P (alc). Each concentration was added in duplicate. The wells infected with virus in the absence of any drug were labeled as “virus only” and the cells not infected with any virus were labeled as “cell only.” The plates were incubated at 37° C. in humidified chambers containing 5% CO₂. After three days, the culture supernatants were transferred into clean conical tubes and subjected to centrifugation at low speed to remove cell debris. The purified supernatants were stored at −135° C. until used. RNA was extracted using a commercial RNA extraction kit purchased from Epicentre Technologies (Madison, WI). The procedure was performed according to the manufacturer's instructions, and samples were stored at −20° C. The level of viral RNA in each sample was determined using the RiboGreen® RNA quantitative kit (Molecular Probes, Eugene, Oreg.) according the manufacturer's instructions.

TABLE 10
Effect of EB fractions on viral RNA

| | Drug Concentration | RNA Concentration |
|---|---|---|
| Samples | (μg/ml) | (ng/ml) |
| Blank (no viral infection) | 0 | — |
| Ribavirin | 1 | 450 |
| Ribavirin | 20 | 225 |
| ZX-2227P (alc) | 1 | 310 |
| ZX-2227P (alc) | 20 | 60 |

Results from the experiments summarized in Table 10 show a reduction in RNA with increasing amounts of ZX-2227P (alc) or ribavirin. The EB fraction ZX-2227P (alc) and ribavirin reduced viral RNA 5- and 2-fold respectively, when the drug concentration was increased from 1 to 20 μg/ml.

In conclusion, the EB fraction ZX-2227P (alc) was more potent in reducing viral RNA production than ribavirin. The observation presented here verified the results of the CPE assays described above, where reduction in cell death was observed in the presence of EB therapeutics. From this experiment we can further conclude that the reduction in cell death, with an increase in the amount of the compounds, is indeed due to the reduction in the amount of virus released from infected cells.

### Example 9

### Isolation, Chemical Structure Determination of Compounds From EB Fractions

The dried and ground root material (100 g) was macerated in 300 ml distilled water at 60-70° C. for 1 h. Then, the macerate was filtered, and the process was repeated 3 times with 250 ml water each with maceration times of 30 min. The combined filtrates were freeze-dried in a lyophilizer to yield 22.58 g of a bright orange powder. To isolate crude compound, the lyophilized water extract (1.00 g) was dissolved in 5.0 ml methanol/water (4/1 v/v). To this solution 4 g of silica gel 60 (63-200 μm, Sorbent Technologies, Atlanta, Ga.) were added, and the mixture was dried under vacuum to yield 5.0 g of homogeneous alkaloid-loaded silica gel ready for application in a vacuum liquid chromatography (VLC) system. The VLC column was prepared with a slurry of 45 g silica gel 60 (TLC grade, Macherey & Nagel, Düren, Germany) in 200 ml n-butanol/water/glacial acetic acid (70/5/10), which was poured into a VLC column 50 mm i.d.×200 mm length. The slurry was allowed to produce a sediment under slight vacuum. After application of the dried alkaloid-loaded silica, stepwise gradient elution was performed using n-butanol/water/glacial acetic acid (70/5/10) (100 ml), n-butanol/water/glacial acetic acid (70/10/10) (300 ml) and n-butanol/water/glacial acetic acid (70/20/10) (300 ml). Fractions of 50 ml each were collected. The chromatographic process was monitored by UV at 366 nm and fractions 7-9 were combined and dried to yield 550 mg of crude compound (ca. 90%).

The pure compound was obtained as follows: an aliquot of 100 mg of the crude compound was dissolved in 1.0 ml of distilled water and added to 3.0 ml of a hot saturated solution of sodium chloride. The mixture was boiled for ca. 1 min and cooled in an ice bath followed by filtration. The compound chloride was precipitated, washed with ice water (1.0 ml) and with ice-cooled ethanol (1.0 ml) to yield 75 mg pure compound chloride as determined by TLC, a single HPLC peak, and proton NMR. Using this method, compound CJ4-16-4 (3,5 dicaffeoyl-quinic acid) and CJ2-27-4 (4,5 dicaffeoyl-quinic acid) were isolated and identified from EB Fraction ZX-2227.

### Example 10

### Antiviral Evaluation of 3,5 Dicaffeoyl-Quinic Acid In Vitro

To determine if CJ4-16-4 (3,5 dicaffeoyl-quinic acid) is responsible for the anti-RSV properties of EB fraction ZX-2227, in vitro testing was performed. 3,5 dicaffeoyl-quinic acid (purity ranges between 80-90%) inhibits RSV-Long with EC₅₀ value of about 0.068 μM in Hep 2 cells with no significant cytotoxicity. In addition, this compound is specific to RSV because it is active against multiple RSV strains, but inactive against other viruses such as Influenza A, HSV-1 and HSV-2. In cotton rat model of RSV infection, it inhibits RSV infection by over one log at 12.5 mg/kg/day.

Antiviral activity and cytotoxicity evaluation in Hep 2 cells was performed as follows. The anti-RSV activities of EB fraction ZX-2227 was determined at different stages of processing using the CPE assay system. Table 11 shows improvement of the anti-RSV properties of EB fraction ZX-2227 due to purification of active compound CJ4-16-4.

TABLE 11
Anti-RSV improvement due to isolation of compound CJ4-16-4 from EB
fraction ZX-2227

| | | EC₅₀ |
|---|---|---|
| Stage | Processing Procedure | (μg/ml) |
| I | Crude Extract (Hot water extract) | 35 |
| II | Extract (Chemical Isolation) | 4.7 |
| III | Extract Fraction: 30-50% single compound; CJVLC.V15 | 0.5 |
| | (Column Separation) | |
| IV | Active Compound, CJ 4-16-4 (80-90% Pure) | 0.035 |
| | from CJVLC.V15 | |

| | EC₅₀ |
|---|---|
| Stage | (μM)* |
| I | 68 |
| II | 9.0 |
| III | 1.0 |
| IV | 0.068 |

*Values are EC₅₀ expressed in μM using the estimated FW-516 of the active compound (CJ 4-16-4).

The cytotoxicity of compound CJ4-16-4 was also evaluated using multiple cell lines. Table 12 shows that minimal cytotoxicity was observed using compound CJ4-16-4.

TABLE 12
Cytotoxicity of CJ4-16-4 using the CPE assay
system using multiple cell lines

| Cell lines | CC₅₀-μg/ml (μM)* |
|---|---|
| Hep 2 | >200 (>400) |
| Vero | >200 (>400) |
| MDCK | >200 (>400) |

*Values in ( ) are CC₅₀ expressed in μM using the estimated FW-516 of the compound.

Next, the anti-RSV properties of CJ4-16-4 were evaluated using multiple RSV strains. Hep-2 cells were used in the CPE assay to determine anti-RSV properties. Table 13 shows that the anti-RSV effects of CJ4-16-4 are not limited to a single strain of RSV. Thus CJ4-16-4 will be used as a therapeutic for treatment of all strains of RSV.

TABLE 13
Effect of CJ 4-16-4 on Multiple RSV Strains

| RSV-Strains | EC₅₀-μg/ml (μM)* |
|---|---|
| RSV-Long | 0.035 (0.068) |
| RSV-Long (Cotton Rat adapted strain) | 1.4 (2.7) |
| RSV-B (Washington) | 0.086 (0.167) |
| RSV-A2 | 0.084 (0.163) |
| RSV-A6 57754 | 0.08 (0.16) |

*Values in ( ) are EC₅₀ expressed in μM using the estimated FW-516 of the compound.

The specificity of the anti-RSV properties of CJ4-16-4 was also evaluated. CJ4-16-4 was tested using influenza type A, herpes simplex virus type 1, and herpes simplex virus type 2 using the CPE assay with MDCK or Vero cells. Table 14 shows that the anti-RSV properties of CJ4-16-4 are specific for RSV.

TABLE 14
Specificity of CJ 4-16-4

| Viruses | EC₅₀-μg/ml (μM)* |
|---|---|
| Influenza A-Shangdong/09/93 (H3N2) | >50 (>100) |
| Herpes simplex virus type 1 (MacIntyre strain) | >50 (>100) |
| Herpes simplex virus type 2 (MS-strain) | >50 (>100) |

*Values in ( ) are EC₅₀ expressed in μM using the estimated FW-516 of the compound.

Mechanism of action studies were performed as follows. Mechanism of action studies were performed using CJ4-16-4 in a CPE assay with Hep-2 cells. Table 15 demonstrates that the time of addition of CJ4-16-4 did not disrupt the anti-RSV effects. Thus, CJ4-16-4 will be used as a treatment for RSV after RSV infection.

TABLE 15
Time of Addition (TOA) of CJ 4-16-4

| | EC₅₀ - μg/ml (μM)* | | | | | |
|---|---|---|---|---|---|---|
| | 0-Hr-Post- | 2-Hr-Post- | 4-Hr-Post- | 6-Hr-Post- | 8-Hr-Post- | 24-Hr-Post- |
| Compound | infection | infection | infection | infection | infection | infection |
| CJ 4-16-4 | 0.051 (0.099) | 0.036 (0.07) | 0.039 (0.076) | 0.043 (0.083) | 0.039 (0.076) | 0.069 (0.13) |

*Values in ( ) are EC₅₀ expressed in μM using the estimated FW-516 of the compound.

### Example 11

### Antiviral Evaluation of 3,5 Dicaffeoyl-Quinic Acid In Vivo

The antiviral activity CJ4-16-4 (3,5 dicaffeoyl-quinic acid) was evaluated in cotton rats using the method described in Example 6. Table 16 and Table 17 list the protocol for in vivo testing and the schedule of administration, respectively.

TABLE 16
Protocol for in vivo testing

| | | | Days of | Route | |
|---|---|---|---|---|---|
| | | Dose | inocu- | of | Day of CR |
| Treatment | Vehicle | Mg/kg/day | lation | delivery | harvest |
| Placebo | 50% DMSO | 0 | D0-D3 | I.P. | 4 |
| CJVLC.V15* | 50% DMSO | 100 B.I.D | D0-D3 | I.P. | 4 |
| CJ-4-16-4 | 50% DMSO | 25 B.I.D | D0-D3 | I.P. | 4 |
| CJ-4-16-4 | 50% DMSO | 12.5 B.I.D | D0-D3 | I.P. | 4 |
| Ribavirin | Water | 90 B.I.D | D0-D3 | I.P. | 4 |

D = day,
CR = cotton rats,
I.P. = intraperitoneally,
B.I.D = in divided doses.
*CJVLC.V15 contains about 30-50% of the single compound CJ4-16-4.

TABLE 17
Schedule for in vivo testing

| Day 0 | Day 1 | Day 2 | Day 3 | Day 4 |
|---|---|---|---|---|
| CR treated I.P. with test | CR treated I.P. | CR treated I.P. with | CR treated I.P. with | Harvest and test CR |
| articles or placebo. | with test articles or | test articles or | test articles or | lungs for RSV. |
| Inoculate the CR with | placebo. | placebo. | placebo. | |
| virus I.N. 30 minutes | | | | |
| later | | | | |

CR = cotton rat,
I.P. = intraperitoneally,
I.N. = intranasally.

Table 18 lists the results of the in vivo testing of CJ4-16-4; specifically, CJ4-16-4 reduced the RSV viral titer in the lungs of infected cotton rats. Thus, these results support the use of CJ4-16-4 (3,5 dicaffeoyl-quinic acid) therapeutically in vivo.

TABLE 18
Anti-RSV properties of CJ4-16-4 are observed in vivo

| | RSV Pulmonary | | | | | |
|---|---|---|---|---|---|---|
| | Titer (log10/ | | | | | |
| | g lung) in CR | | | | | Reduction |
| Treatment (Dose) | A | B | C | D | Mean ± SD | (Log 10) |
| Placebo | 4.3 | 4.8 | 4.3 | 4.3 | 4.4 ± 0.3 | 0 |
| CJVLC.V15* | 0 | 2.8 | 0 | 3.3 | 1.5 ± 1.8 | 2.9 |
| (100 mg/kg/day) | | | | | | |
| CJ4-16-4 (25 mg/kg/day) | 2.8 | 3.3 | 3.3 | 3.3 | 3.2 ± 0.3 | 1.2 |
| CJ4-16-4 (12.5 mg/kg/day) | 3.3 | 3.3 | 3.3 | 3.3 | 3.3 ± 0.0 | 1.1 |
| Ribavirin (90 mg/kg/day) | 2.8 | 3.3 | 0 | 0 | 1.5 ± 1.8 | 2.9 |

SD = Standard Deviation and
CR = cotton rats;
*CJVLC.V15 contains about 30-50% of the single compound CJ 4-16-4.

### Example 12

### Antiviral Evaluation of 4,5 Dicaffeoyl-Quinic Acid In Vitro

The other compound CJ2-27-4 (4,5 dicaffeoyl quinic acid) isolated from EB fraction ZX-2227 was also evaluated. First, the anti-RSV properties of CJ2-27-4 were evaluated using multiple RSV strains. Hep-2 cells were used in the CPE assay to determine anti-RSV properties. Table 19 shows that the anti-RSV effects of CJ2-27-4 are not limited to a single strain of RSV. Thus, CJ2-27-4 will be used as a therapeutic for treatment of all strains of RSV.

TABLE 19
Effect of CJ2-27-4 on Multiple RSV Strains

| RSV-Strains | EC₅₀-μg/ml |
|---|---|
| RSV-Long | 0.052 |
| RSV-Long (Cotton Rat adapted strain) | 5.519 |
| RSV-B (Washington) | 0.218 |
| RSV-A2 | 0.521 |

The cytotoxicity of compound CJ2-27-4 was also evaluated using multiple cell lines. Table 20 shows that minimal cytotoxicity was observed using compound CJ2-27-4.

TABLE 20
Cytotoxicity of CJ 2-27-4 using CPE assay system

| Cell lines | CC₅₀-μg/ml |
|---|---|
| Hep 2 | >200 |
| Vero | >200 |
| MDCK | >200 |

The specificity of the anti-RSV properties of CJ2-27-4 was also evaluated. CJ2-27-4 was tested using influenza type A, herpes simplex virus type 1, and herpes simplex virus type 2 using the CPE assay with MDCK or Vero cells. Table 21 shows that the anti-RSV properties of CJ2-27-4 are specific for RSV. Thus CJ2-27-4 (4,5 dicaffeoyl quinic acid) will be used as a therapeutic for RSV infection in vivo.

TABLE 21
Specificity of CJ 2-27-4

| Viruses | EC₅₀-μg/ml |
|---|---|
| Influenza A-Shangdong/09/93 (H3N2) | >50 |
| Herpes simplex virus type 1 (MacIntyre strain) | >50 |
| Herpes simplex virus type 2 (MS-strain) | >50 |

### Example 13

### Identification of Transesterification of 3,5 Dicaffeoyl-Quinic Acid and Derivatives Thereof that Resist Transesterification

The stability of 3,5 Dicaffeoyl-Quinic Acid (3,5-DCQA) was first evaluated in methanol. 3.8 mg 3,5-DCQA was dissolved in 0.2375 ml MeOH (conc.=16 mg/ml), and then diluted as ½, ¼ and ⅛ dilution, respectively. All samples were subjected to HPLC in 0 Day, 1 Day, 2 Day and 4 Day, and the Area% of 3,5-DCQA was calculated.

TABLE 22
Degradation of 3,5-DCQA in methanol

| Time | Stock (16 mg/ml) | ½ dilution | ¼ dilution | ⅛ dilution |
|---|---|---|---|---|
| 0 Day | 97.40 | | | 97.32 |
| 1 Day | 97.16 | | | 96.84 |
| 2 Day | 96.42 | 97.03 | 96.65 | 94.44 |
| 4 Day | 95.39 | 95.66 | 95.88 | 94.27 |

Next, the stability of 3,5-DCQA was evaluated in various concentrations of DMSO. 5.2 mg 3,5-DCQA was dissolved in 0.208 ml DMSO (conc.=25 mg/ml), and then diluted with water as 40% DMSO in water solution, 20% DMSO in water solution, and 10% DMSO in water solution, respectively. All samples were subjected on HPLC in 0 Day, 1 Day, 2 Day and 4 Day, and the Area % of 3,5-DCQA was calculated. Table 23 summarizes these results. No statistically significant degradation of 3,5-DCQA was observed in 100% DMSO; however, increasing amounts of 3,5-DCQA degradation was observed in increasing concentrations of water.

TABLE 23
Degradation of 3,5-DCQA in DMSO

| | 100% DMSO | 40% DMSO | 20% DMSO in | 10% DMSO in |
|---|---|---|---|---|
| Time | (25 mg/ml) | in H₂O | H₂O | H₂O |
| 0 Day | 97.28 | | | 97.25 |
| 1 Day | 97.34 | 96.33 | 94.56 | 93.60 |
| 2 Day | 97.15 | 94.72 | 91.91 | 89.50 |
| 4 Day | 97.09 | 93.90 | 89.08 | 86.01 |

In the presence of water, 3,5-DCQA was converted into 4,5-dicaffeoylquinic acid (4,5-DCQA), as observed using HPLC. HPLC analysis of 3,5-DCQA in DMSO and H₂O (conc.=2.5 mg/ml) at 0 day, 2 Day and 4 Day indicated a progressive degradation of 3,5-DCQA. By the forth day in 10% DMSO in H₂OP, over 11% of 3,5-DCQA was changed into 4,5-DCQA.

The rate of degradation of 3,5-DCQA was again tested in increasing concentrations of water. 5.4 mg 3,5-DCQA was dissolved in 0.266ml DMSO (conc.=20.3 mg/ml), and then diluted with water to ½, ¼ and ⅛ dilution, respectively. All samples were subjected on HPLC in 0 Day, 1 Day, 2 Day and 4 Day, and Area % of 3,5-DCQA was calculated. A clear increase in the degradation of 3,5-DCQA was observed in increasing concentrations of water. HPLC analysis indicated that by the fourth day in solution, over 11% of 3,5-DCQA had changed into 4,5-DCQA.

TABLE 24
Degradation of 3,5-DCQA in H₂O

| Time | Stock (20.3 mg/ml) | ½ dilution | ¼ dilution | ⅛ dilution |
|---|---|---|---|---|
| 0 Day | 97.05 | | | 96.91 |
| 1 Day | 96.09 | 94.62 | 92.79 | 92.60 |
| 2 Day | 93.94 | 92.71 | 91.06 | 89.02 |
| 4 Day | 91.90 | 90.20 | 88.14 | 85.55 |

These results show that transesterification of 3,5-DCQA into 4,5-DCQA occurs in protic solvents. This problem did not become apparent until inventors observed decreased biological efficacy and solubility upon extended storage in a protic solvent. The investigation into the activity loss revealed that chemical rearrangement of the 3,5-DCQA into 4,5-DCQA accounted for this loss. This transesterification reaction has significant deleterious consequences for clinical applications. In response to this problem, an aprotic solvent (e.g., DMSO) could be used as a carrier for 3,5-DCQA; however, significant toxicity to the subject would be likely result from using many aprotic solvents, and using aprotic solvents would likely prevent the oral administration of this drug in a clinical setting. The spontaneous inactivation and solubility reduction of 3,5-DCQA underscore the significance of the chemical modifications presented herein that result in 3,5-DCQA derivatives that exhibit increased stability.

In response to the problem of transesterification of 3,5-DCQA into 4,5-DCQA, the inventors have proposed the use of derivatives of 3,5-DCQA that will not undergo this transesterification reaction. These derivatives may then be used to treat RSV infection. These derivatives include compounds having the following structure:

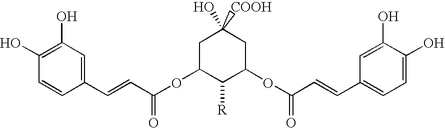

R may be chosen from the group consisting of alkyl, alkenyl, alkynyl, alkoxy, alkylaryl, halogen, hydrogen, OH, trihalomethyl, NO₂, thioether, amine, SH, and NH₂. In certain preferred embodiments, R is F, Cl, SH, NH₂, —OCH₃, —OCH₂ (CH₂)ₙ CH₃, —(CH₂)ₙ OH, —(CH₂)ₙ NH₂, —N(CH₂)ₙ OH, —OCOOC(CH₃)₃, or an amino acid.

### Example 14

### Preliminary Results on the Synthesis of CJ4-16-4 ((-)-3,5-Di-O-caffeoylquinic Acid 9)

The inventors have developed a synthetic scheme for production of CJ 4-16-4 and have successfully completed the synthesis up to the intermediate 13 as indicated in the synthetic scheme shown below. The detailed experimental procedures are described below.

From (−)-quinic acid 1: Bicyclic γ-lactone 6, commonly called quinide, is formed by heating (−)-quinic acid 1 at 230 ° C. in an open flask (Joseph et al., 1964). The selective protection of the C-4 hydroxy group of 6 is achieved using tert-butyldimethylsilyl (TBDMS) chloride in N,N-dimethylformamide containing tetrabutylammonium iodide and triethylamine at 90° C. for 24 h (Manthey et al., 1997). Treatment of 12 with aqueous base affords the parent acid 13. We are now investigating the caffeoylation of the TBDMS derivative 13 to form 14 (Sefkow, 2001) and its subsequent deacetylation and desilylation to form our target molecule 9.

Since the isolation of compound 13 is not trivial, we are also investigating its caffeoylation without physically isolating compound 13. Promising results are obtained by freeze-drying the mixture of hydrolysis of compound 12 and subsequent direct caffeoylation.

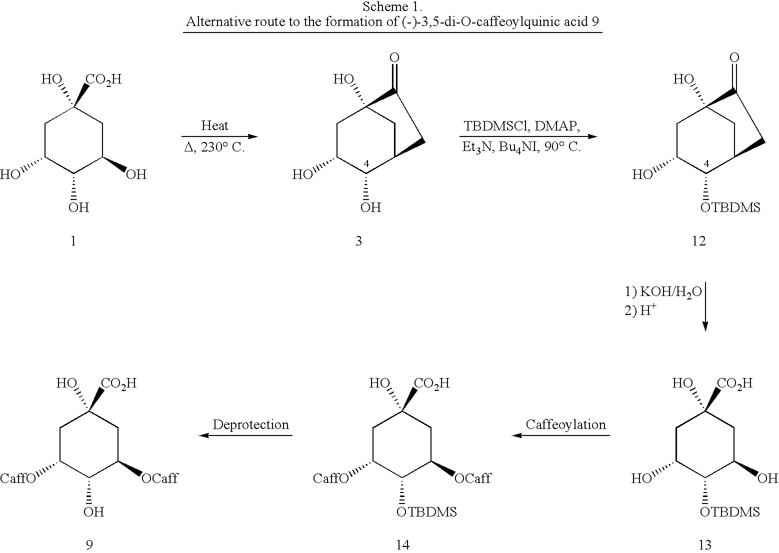

Preparation of (−)-Quinide 6: (−)-Quinic acid 1, 20 g, was heated at 230° C. for 15 min. in an open flask. After cooling the glassy residue was recrystallized from methanol/acetone (v/v=9:1) to afford 8.5 g (47%) of (−)-quinide 6 δ_{H} (400 MHz; CD₃OD) 4.76 (1H, t, J 5.6, 3-H), 4.03 (1H, t, J 4.4, 4-H), 3.76 (1H, ddd, J 4.4, 6 and 11.2, 5-H), 2.51 (1H, d, J 11.6, 6ax-H), 2.28 (1H, ddd, J 2.4, 5.6 and 11.6, 6eq-H), 2.08 (1H, ddd, J2.8, 6.4 and 11.6, 2eq-H) and 1.93 (1H, t, J 11.6, 2ax-H); δ_{C} (100 MHz; CD₃OD) 179.6, 77.9, 73.2, 67.4, 66.8, 40.1 and 37.9.

Two alternative procedures are being examined with a view to increasing yield: (i) using TsOH in refluxing toluene, and (ii) using Amberlite IR-120 (H⁺) in refluxing DMF/benzene.

Preparation of 4-tert-Butyldimethylsiloxy-1,5-dihydroxycyclohexane-1,3-carbolactone 12: To a stirred solution of (−)-quinide 6 (104 mg, 0.35 mmol), DMAP (24 mg, 0.20 mmol) and tetrabutylammonium iodide (11 mg, 0.03 mmol) in dry DMF (2.0 ml) and triethylamine (0.1 ml) at room temperature under argon was added 104 mg (0.69 mmol) tert-butyldimethylsilyl chloride. The resultant solution was heated at 90° C. for 24 h, and after cooling was diluted with acetone (10 ml) and filtered. The solution was mixed with 9 g silica gel (TLC grade) and subjected to VLC column eluting with hexane/EtOAc (v/v=3:1) to yield 53.8 mg (31%) of 4-tert-butyldimethylsiloxy-1,5-dihydroxycyclohexane-1,3-carbolactone 12 and 64.6 mg (37%) of 5-tert-butyldimethylsiloxy-1,4-dihydroxy cyclohexane-1,3-carbolactone as white needles. δ_{H} (400 MHz; CDCl₃) 4.66 (1H, t, J4.8, 3-H), 4.10 (1H, t, J4.0, 4-H), 3.81 (1H, ddd, J 4.0, 6.8 and 10.8, 5-H), 2.51 (1H, d, J 11.2, 2ax-H), 2.32 (1H, m, 2eq-H), 2.21 (1H, m, 6eq-H), 1.86 (1H, t, J 11.6, 6ax-H), 0.92 [9H, s, C(CH₃)₃], 0.15 (3H, s, SiCH₃) and 0.12 (3H, s, SiCH₃); δ_{C} (100 MHz; CDCl₃) 177.8 (C), 76.5 (CH), 72.0 (C), 66.8 (CH), 66.1 (CH), 40.4 (CH₂), 36.4 (CH₂), 25.7 [C(CH₃)₃], 18.0 (C), −4.6 (SiCH₃) and −4.7 (SiCH₃).

The inventor is currently optimizing conditions that would give a higher proportion of the 4-O-protected derivative 12. Please note that the 5-O-protected isomer can be recycled, i.e., the loss of material is minimized.

Preparation of 4-tert-Butyldimethylsiloxy-1,3,5-trihydroxycyclohexanecarboxylic acid 13: A mixture of 4-tert-butyldimethylsiloxy-1,5-dihydroxycyclohexane-1,3-carbolactone 12 (150 mg, 0.52 mmol) and KOH (1.5 ml 0.4M) was stirred at room temperature. After 25 minutes, 0.01 ml 1.4M HOAc was added to convert the potassium salt into the parent acid 13 and the stirring was continued at room temperature for 35 min. The solution was extracted with EtOAc (3×5 ml), the combined organic extracts were dried over MgSO₄, filtered, and the solvents were removed under reduced pressure. The residue comprises 4-tert-butyldimethylsiloxy-1,3,5-trihydroxycyclohexanecarboxylic acid 13. δ_{H} (400 MHz; CD₃OD) 4.11 (1H, br. s, 3-H), 3.85 (1H, t, 4-H), 3.59 (1H, br.s, 5-H), 2.09-1.86 (4H, m, 2-H₂ and 6-H₂), 0.90 [9H, s, C(CH₃)₃], 0.10 (3H, s, SiCH₃) and 0.09 (3H, s, SiCH₃).

All of the compositions and methods disclosed and claimed herein can be made and executed without undue experimentation in light of the present disclosure. While the compositions and methods of this invention have been described in terms of preferred embodiments, it will be apparent to those of skill in the art that variations may be applied to the compositions and methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
U.S. Pat. No. 5,211,944
U.S. Pat. No. 5,494,661
U.S. patent appln. 2002/0111382
Ardrey, Spectroscopy Europe, 4:10-18, 1992.
Beneke, In: Human Mycoses, Upjohn Co., Kalamazoo, Mich., 1979.
Chanock and McIntosh, In: Respiratory syncytial virus, Fields et al., (Eds.), Fields Virology, 2^{nd} Ed., Raven Press, NY, 1045-1074, 1990.
Cotton, Ethnobotany-Principles and Applications, pub. John Wiley, Chichester, 1996.
Cragg et al., Ciba Found Symp., 185:178-190, 1994.
Dowell et al., J. Infect. Dis., 174:456-462, 1996.
Fenn et al., J. Phys. Chem., 88:4451-4459, 1984.
Glezen et al., In: Viral Infections of Humans: Epidemiology and Control, Evans (Ed.), Plenum Press, NY, 337-349, 1982.
Joseph et al., J. Org. Chem., 29:3596-3598, 1964.
Locher et al., J. Ethnopharmacol., 49(1):23-32, 1995.
Manthey et al., J. Chem. Soc., 1:625-628, 1997.
McCloskey, In: Mass Spectrometry, Academic Press, NY, Vol. 193, 1990.
Smith, In: Opportunistic Mycoses of Man and Other Animals, CAB Intl., Wallingford, UK, 1989.
Parrott et al., Am. J. Epidemiol., 98, 289-300, 1973.
PCT Appln. WO 90/14148
Principe, In: Conservation of Medicinal Plants, Akerele et al. (Eds.), Heywood and Synge, 1991
Remington's Pharmaceutical Sciences, 18th Ed. Mack Printing Company, 1289-1329, 1990.
Scripp's Antifungal Report, by PJB Publications Ltd, 1992
Sefkow et al., Eur. J. Org. Chem., 2735-2742, 2001.
Smith et al., Anal. Chem. 62, 882-89, 1990.
Wolfe, Med. Clin. North Am., 66(3):707-720, 1982.

What is claimed is:

1. A method of identifying a therapeutic composition comprising:
(a) identifying a substance that has been used as an ethnobotanical remedy for a given disease state;
(b) subjecting said substance to fractionation;
(c) testing one or more fractions from step (b) in a screen for activity against said given disease state; and
(d) identifying one or more fractions that exhibit activity against said disease state,
wherein an identified fraction in step (d) comprises a therapeutic composition against said given disease state.

2. The method of claim 1, wherein said fractionation comprises one or more of freezing, grinding, solubilizing, extracting, eluting, filtering, precipitating, partitioning or chromatography.

3. The method of claim 1, wherein said disease state comprises an infectious disease.

4. The method of claim 3, wherein said infectious disease is a viral disease, a bacterial disease or a fungal disease.

5. The method of claim 1, wherein said disease state is a genetic disease.

6. The method of claim 1, wherein said disease state comprises cancer.

7. The method of claim 1, wherein said disease state comprises a disease selected from the group consisting of cardiac disease, diabetes, a neurodegenerative disease, a viral disease, a fungal disease, a bacterial disease, and cancer.

8. The method of claim 1, wherein said ethnobotanical comprises a quinic acid derivative.

9. The method of claim 1, wherein said testing comprises an in vitro assay.

10. The method of claim 1, further comprising identifying an active compound in said fraction.

11. A purified compound having the structure: 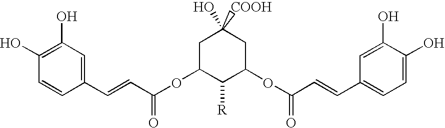
wherein R is selected from the group consisting of alkyl, alkenyl, alkynyl, alkoxy, alkylaryl, halogen, hydrogen, trihalomethyl, NO₂, thioether, amine, SH, NH₂, —OCH₃, —OCH₂ (CH₂)ₙ CH₃, —(CH₂)ₙ OH, —(CH₂)ₘ NH₂, —N(CH₂)ₙ OH, —OCOOC(CH₃)₃, and an amino acid;
wherein n=0, 1,2, 3,4, or 5.

12. The compound of claim 11, wherein R is a halogen.

13. The compound of claim 12, wherein R is —F or —Cl.

14. The compound of claim 11, wherein R is selected from the group consisting of: SH, NH₂, —OCH₃, —OCH₂ (CH₂)ₙ CH₃, —(CH₂)ₙ OH, —(CH₂)ₙ NH₂, —N(CH₂)ₙ OH, and —OCOOC(CH₃)₃; wherein n=0,1,2,3,4, or 5.

15. A method of inhibiting respiratory syncytial virus (RSV)-induced cytotoxicity comprising contacting a cell infected with RSV with the compound of claim 11.

16. The method of claim 15, wherein said cell is in an animal subject.

17. The method of claim 16, wherein said compound is administered to said animal intranasally, intradermally, intraarterially, intraperitoneally, intralesionally, intracranially, intraarticularly, intraprostaticaly, intrapleurally, intratracheally, intranasally, intravitreally, intravaginally, intrarectally, topically, intratumorally, intramuscularly, intraperitoneally, subcutaneously, subconjunctival, intravesicularlly, mucosally, intrapericardially, intraumbilically, intraocularally, orally, topically, locally, inhalation (e.g., aerosol inhalation), injection, infusion, continuous infusion, localized perfusion bathing target cells directly, via a catheter, via a lavage, in cremes, in lipid compositions (e.g., liposomes), or by other method or any combination of the forgoing as would be known to one of ordinary skill in the art.

18. The method of claim 16, wherein said compound is administered in a pharmaceutically acceptable carrier, diluent or vehicle.

19. The method of claim 16, further comprising administering to said animal a second anti-viral composition.

20. A method of inhibiting respiratory syncytial virus (RSV)-induced cytotoxicity comprising contacting a cell infected with RSV with a compound having the structure of the compound of claim 11.

21. The method of claim 20, wherein said cell is in an animal subject.

22. The method of claim 21, wherein said compound is administered to said animal intranasally, intradermally, intraarterially, intraperitoneally, intralesionally, intracranially, intraarticularly, intraprostaticaly, intrapleurally, intratracheally, intranasally, intravitreally, intravaginally, intrarectally, topically, intratumorally, intramuscularly, intraperitoneally, subcutaneously, subconjunctival, intravesicularlly, mucosally, intrapericardially, intraumbilically, intraocularally, orally, topically, locally, inhalation (e.g., aerosol inhalation), injection, infusion, continuous infusion, localized perfusion bathing target cells directly, via a catheter, via a lavage, in cremes, in lipid compositions (e.g., liposomes), or by other method or any combination of the forgoing as would be known to one of ordinary skill in the art.

23. The method of claim 21, wherein said compound is administered in a pharmaceutically acceptable carrier, diluent or vehicle.

24. The method of claim 20, further comprising administering to said animal a second anti-viral composition.
